(19) United States
(12) Patent Application Publication
(10) Pub. No.: US 2014/0274927 A1
(43) Pub. Date: Sep. 18, 2014

(54) TNF-Related Biomarkers For Assessing Cancer Cell Response To Treatment With Taxane And/Or Anthracycline Drugs

(71) Applicants: Amadeo Mark Parissenti, Sudbury (CA); Jason A. Sprowl, Cordova, TN (US)

(72) Inventors: Amadeo Mark Parissenti, Sudbury (CA); Jason A. Sprowl, Cordova, TN (US)

(21) Appl. No.: 14/147,030

(22) Filed: Jan. 3, 2014

Related U.S. Application Data
(60) Provisional application No. 61/749,104, filed on Jan. 4, 2013.

(51) Int. Cl. G01N 33/574; C12Q 1/68

(52) U.S. Cl. CPC G01N 33/57484; G01N 33/57415; G01N 33/57449; C12Q 1/6886
USPC 514/34; 435/7.92; 514/449; 435/7.1; 435/6.12; 506/9

(57) ABSTRACT

A method of evaluating response to a taxane drug and/or anthracycline drug, the method comprising measuring a level of a TNF biomarker in a biological sample comprising the cancer cell after contacting the cancer cell with the taxane drug and/or anthracycline drug.

### RELATED APPLICATIONS

This application is a U.S. regular application which claims priority from U.S. Provisional patent application Ser. No. 61/749,104 filed Jan. 4, 2013, which application is incorporated herein in its entirety by reference.

### SUBMISSION OF SEQUENCE LISTING

The Sequence Listing associated with this application is filed in electronic format via EFS-Web and is hereby incorporated into the specification in its entirety. The name of the text file containing the Sequence Listing is “132175 Sequence Listing.” The size of the text file is 5.47 KB, and the text file was created on Jan. 2, 2014.

### FIELD

The disclosure relates to methods for assessing cancer cell response to a taxane drug and/or an anthracycline drug containing regimen using TNF biomarkers and particularly to methods for assessing patient response to a taxane drug and/or an anthracycline drug containing regimen using TNF biomarkers such as TNFalpha.

### INTRODUCTION

Breast cancer is the most common neoplasm for women in the western world, with mortality rates second only to lung cancer [1]. Surgery is the primary treatment for most breast cancers (in North America), followed by radiation therapy and/or systemic adjuvant chemotherapy [2]. In locally advanced or inflammatory forms of the disease, neoadjuvant chemotherapy is employed to shrink tumours and improve local control prior to surgery, after which additional rounds of chemotherapy are administered [3;4]. Ovarian cancer is the 8th most prevelant cancer in women [1], but effective treatment remains elusive [5], as more than half of patients succumb to the disease within 5 years [6]. Treatment of ovarian cancer involves surgical removal of the tumour followed by adjuvant chemotherapy, although neoadjuvant chemotherapy followed by interval debulking appears to be gaining favor recently [7]. Two popular classes of cytotoxic drugs used in breast cancer treatment (in the adjuvant and neoadjuvant settings) include the anthracyclines (typically doxorubicin or epirubicin) and the taxanes (e.g. paclitaxel or docetaxel) [8]. Anthracyclines inhibit topoisomerase 2 [9], intercalate between DNA strands [10], and cause DNA lesions [11]. Taxanes, on the other hand, block microtubule depolymerization, inducing cell cycle arrest at mitosis and cell multinucleation [12;13]. In breast cancer, taxanes are typically administered after anthracyclines and greatly improve clinical response [8]. However, administration of dose dense taxanes prior to anthracycline regimens is becoming increasingly common [14]. In ovarian cancer, the taxane paclitaxel is typically administered in combination with a platinating agent (carboplatin) [15].

Clinical resistance to taxanes and other drugs can be innate (residing within tumours prior to chemotherapy) or acquired upon drug exposure. Many mechanisms for acquired drug resistance have been identified in vitro, including the overexpression of drug transporters such as Abcb1, which actively transport these drugs outside of tumour cells [16]. Abcb1 expression is only induced at high drug concentrations, where it can promote 100- to >10.000-fold resistance to taxanes in vitro [17;18]. Tumours typically only “see” a small fraction of the drug available within the vascular system [19;20], it is doubtful that taxane concentrations in tumours reach levels sufficiently high to induce Abcb1 expression. Supporting this view, tumour expression of any drug transporter cannot be used to reliably identify taxane-resistant tumours [21], nor have inhibitors of drug transporters proven successful in blocking taxane resistance in cancer patients [22;23]. Multiple mechanisms of taxane resistance likely occur simultaneously in tumours [24].

It has been observed that activation of NF-kB (i.e. nuclear NF-kB/p65 staining in pre-therapy specimens) was linked to chemoresistance. [48].

While a reduction in tumour size by palpation or MRI (partial clinical response) or the complete absence of disease as detected by such methods (complete clinical response) are common in advanced breast cancer patients after chemotherapy, such responses are typically short-lived, with evidence of disease post-treatment [25]. However, if a pathologic complete response (pCR) (e.g. complete eradication of all living tumour cells in the breast and axilla) is observed post-treatment, such patients have a considerably lower incidence of disease recurrence and substantially longer progression-free and overall survival [25].

A recent analysis of the findings of 28 clinical trials revealed that the pCR rate for patients with locally advanced breast cancer being treated with various non-trastuzumab-based chemotherapy regimens was 16.4±1.3%, suggesting that ˜84% of patients had residual disease post-treatment [26]. Moreover, the SWOG-8814 clinical trial reported in a 10 year follow-up study that only 8% of patients received an overall survival benefit from chemotherapy with cyclophosphamide, adriamycin (doxorubicin), 5-Fluorouracil and tamoxifen (CAF-T) compared to patients administered tamoxifen alone [26]. Similarly, only a small percentage of ovarian cancer patients receive a survival benefit from adjuvant or neoadjuvant chemotherapy, in particular for patients with early stage disease [27].

In contrast to the small number of breast and ovarian cancer patients that receive a survival benefit from taxane-based chemotherapy, many patients experience significant toxicities from this therapy, including neutropenia, emesis, anemia, thrombocytopenia, neuropathy, shortness of breath, severe fatigue, infertility, and rarely premature death [28-30]. Thus, there is an unmet need for a reliable test(s) to help the oncologist identify which patients are likely to receive a survival benefit from taxane-based chemotherapy (a pCR) and which will not. Using such a test, non-responders could be spared the toxicities associated with the ineffective regimen, and/or moved quickly to other potentially more beneficial treatments, including surgery, radiation therapy, or other chemotherapy regimens. Valuable health care dollars could also be saved by reducing the costs associated with the administration of ineffective chemotherapy drugs and the treatment of chemotherapy-related toxicities in patients.

MRI scans can assess changes in tumour size during treatment, but cannot determine whether surviving tumours are viable. Reductions in ¹⁸F-deoxy-glucose uptake by tumours (measured by PET scans) often correlate with achievement of pCR in patients [34-36], but sensitivity is only 23% in common lesions of <10 mm [37] and in well-differentiated tumours [38;39]. Since the protein CA-125 is highly expressed in some ovarian cancers, it has been used to monitor response to chemotherapy and to detect early disease recurrence in ovarian cancer patients [40]. However, a 15 year retrospective study revealed that about half of women with early stage ovarian cancer lack elevated CA-125 levels [41;42] and CA-125 levels can be elevated in patients with non-malignant conditions [43]. Thus, CA-125 cannot widely be used to reliably monitor chemotherapy response in ovarian cancer.

### SUMMARY

An aspect provides a method of evaluating cancer cell drug response to a taxane drug and/or anthracycline drug, the method comprising:
a. measuring a level of a TNF biomarker in a biological sample comprising the cancer cell after contacting the cancer cell with the taxane drug and/or anthracycline drug;
b. detecting a difference or a lack of difference in the level of the TNF biomarker compared to a control; and
c. scoring the cancer cell drug toxicity/resistance and/or identifying the cancer cell as sensitive or resistant to the to taxane drug and/or anthracycline drug according to the detected difference in the TNF biomarker level.

In an embodiment, the method further comprises the step of contacting the cancer cell with the taxane drug and/or anthracycline drug prior to measuring the level of the TNF biomarker in the biological sample comprising the cancer cell.

In another embodiment, the TNF biomarker is selected from TNFα, active NFκB, and biomarkers listed in Table 2.

In another embodiment, the TNF biomarker is selected from TNFα and active NFκB. In an embodiment, an increased level of TNFα and/or active NFκB compared to the control is indicative of resistance to the taxane drug and/or anthracycline drug.

In another embodiment, an increased level of TNFα compared to the control is indicative of tumor toxicity (e.g. sensitivity) to the taxane drug and/or anthracycline drug and an increased level of active NFκB compared to the control is indicative of resistance to the taxane drug and/or anthracycline drug.

In another embodiment, the cancer cell is a breast cancer cell, an ovarian cancer cell, a sarcoma cell, a lymphoma cell, a leukemic cell, a uterine cancer cell, a colon cancer cell or a lung cancer cell.

In another embodiment, an increased level of a TNF resistance biomarker and/or decreased level of a TNF sensitivity biomarker compared to a control is indicative of resistance of the cancer cell to the taxane drug and an increased level of a TNF sensitivity biomarker or a decreased level of a TNF resistance biomarker compared to a control is indicative of toxicity of the cancer cell to the taxane drug and/or anthracycline drug.

In yet another embodiment, the TNF resistance biomarker is active NFkappaB, TNFR2 and/or a biomarker listed as increased in Table 2 and/or the TNF sensitivity biomarker is TNFalpha, TNFR1 or a biomarker listed as decreased in Table 2.

In an embodiment, wherein the TNF biomarker is TNFalpha, a decreased level or a lack of an increased level of TNFalpha is indicative of resistance.

In an embodiment, the TNFalpha level is determined in combination with TNFR level. In an embodiment, an increase of TNFalpha in the presence of TNFR1 expression is indicative of sensitivity. In another embodiment, an increase of TNFalpha in the presence of decreased TNFR1 is indicative of resistance. In yet another embodiment, an increase in TNFalpha in the presence of increased TNFR2 expression is indicative of resistance.

In another embodiment, an increase in active NFkappaB, and/or in a level of a biomarker listed as increased in Table 2 and/or a decrease in TNFR1 levels, and/or in a level of a biomarker listed as decreased in Table 2, compared to a control is indicative of resistance to taxane drug and/or anthracycline drug.

In yet another embodiment, the control is a biological sample comprising a nonresistant cell, or a resistant cell, and/or is a standard amount or a reference threshold associated with taxane resistance.

In yet another embodiment, the cancer cell is a primary cancer cell.

In yet another embodiment, the primary cancer cell is in vitro.

In another embodiment, the primary cancer cell is in vivo.

Another aspect provides a method of evaluating cancer cell drug response to administration of a taxane drug and/or anthracycline drug in a subject in need thereof, the method comprising:
a. measuring a level of a TNF biomarker in a biological sample comprising pathologic tissue obtained from the subject after administering to the subject of one or more doses of the taxane drug and/or anthracycline drug;
b. detecting a difference or a lack of difference in the relative level of the TNF biomarker compared to a control; and
c. scoring the cancer cell drug toxicity/resistance and/or identifying the cancer cell as sensitive or resistant to the to taxane drug and/or anthracycline drug toxicity according to the detected difference in in the TNF biomarker level.

In an embodiment, the TNF biomarker is TNFα. TNFα is a sensitivity biomarker as its relative level in biological samples comprising pathologic tissue obtained from a subject during and/or after treatment is inidcative of clinical response.

In an embodiment, the method is for evaluating clinical response in a subject afflicted with breast or ovarian cancer, the method comprising:
a. measuring a level of a TNF biomarker, such as TNFα, in a biological sample comprising pathologic tissue obtained from the subject after administering to the subject one or more doses of the taxane drug and/or anthracycline drug;
b. detecting a difference or lack of difference in the level of the TNF biomarker compared to a control; and
c. scoring the clinical response and/or predicting the clinical response for the subject according to the detected difference in in the relative leve of the TNF biomarker;
wherein i) an increase in the relative level of TNFα and/or a TNF sensitivity biomarker is indicative that the subject is positively responding and/or will have a positive clinical response or ii) a lack of increase or a decrease in the level of TNFα and/or a TNF sensitivity biomarker and/or an increase in a level of a TNF resistance biomarker is indicative that the subject is negatively responding and/or will have a poor clinical response.

In an embodiment, the method further comprises administering one or more doses of the taxane drug and/or anthracycline drug to the subject prior to measuring the level of a TNF biomarker in the biological sample.

In one embodiment, the drug is administered systemically.

In another embodiment, the drug is administered directly to the tumour.

In a further embodiment, the TNF resistance biomarker is selected from active NFkappaB, TNFR2 and/or a biomarker listed as increased in Table 2 and/or the TNF sensitivity biomarker is selected from TNFalpha, TNFR1 and/or a biomarker listed as decreased in Table 2.

In an embodiment, the TNF biomarker is TNFα.

In another embodiment, an increase in a TNFα level, optionally in combination with a steady TNFR1 level is predictive of a good clinical response.

In yet another embodiment, the clinical response is progressive disease, stable disease, partial response, complete clinical response or pathological complete response.

In another embodiment, the level of the TNF biomarker is increased at least 2×, 3×, 4×, 5×, 6×, 7×, 8×, 9×, 10×, 11×, 12×, 13×, 14×, 15×, 16×, 17×, 18×, 19×, or at least 20× compared to control.

In another embodiment, the breast cancer is locally advanced breast cancer (LABC), and/or the breast cancer is Her2+, triple negative, basal subtype, luminal A, normal, or luminal B subtype (e.g. luminal B1 or luminal b2). In an embodiment, the breast cancer is invasive breast cancer. In an embodiment, the histologic type of invasive breast cancer is invasive ductal carcinoma, invasive lobular carcinoma, medullary carcinoma or tubular carcinoma. In an embodiment the grade is grade I, II or III. In another embodiment, the breast cancer is locally advanced breast cancer or inflammatory breast cancer. The breast cancer subtype can be also classified according to receptor status including hormone receptor positive, hormone receptor negative, her1+ve, her2−ve or functionally classified as luminal A, luminal b, basal*, TNBC, Her2+ve, or unclassified.

In another embodiment, the ovarian cancer is epithelial, serous, mucinous, endometrioid, clear cell, or undifferentiated/unclassified ovarian cancer.

In another embodiment, the taxane drug is selected from paclitaxel, docetaxel, larotaxel, Abraxane, docoxahexaenoic acid-linked paclitaxel, paclitaxel polyglumex, Ortataxel, Genexol, liposomal-encapsulated paclitaxel, and paclitaxel in a Vitamin E emulsion.

In another embodiment, the anthracycline drug is selected from epirubicin, doxorubicin, epirubicin, daunorubicin, idarubicin, valrubicin, and mitoxantrone.

In yet another embodiment, the TNF biomarker measured is TNF biomarker transcript. In another embodiment, the TNF biomarker measured is TNF biomarker polypeptide.

In yet another embodiment, the TNFalpha measured is TNFalpha transcript. In a further embodiment, the TNFalpha measured is TNFalpha polypeptide.

In another embodiment, the TNF biomarker polypeptide, optionally the TNFalpha polypeptide is measured by enzyme linked immunosorbent assay (ELISA) or tissue microarray.

In another embodiment, the TNF biomarker polypeptide, optionally TNF alpha polypeptide level measured is the relative TNFalpha polypeptide level which is relative to one or more reference standard gene polypeptide levels.

In another embodiment, the TNF biomarker transcript, optionally TNFalpha transcript is measured by polymerase chain reaction (PCR). In an embodiment, the PCR is quantitative PCR. In another embodiment, the PCR is RT-PCR.

In another embodiment, the TNF biomarker transcript, optionally TNF alpha transcript level measured is the relative TNFalpha transcript level which is relative to one or more reference standard gene transcript levels.

In another embodiment, the one or more reference standard genes are selected from HMBS, HPRT1, MRPL19, PUM1, RPL13A, SDHA, and SF3A1. In an embodiment, optionally 3 or more, 4 or more, 5 or more 6 or more or all 7 of said reference genes are selected, optionally wherein the transcript level of said reference genes is measured using a primer set for the corresponding reference standard gene listed in Table 3.

In yet another embodiment, the level of the TNF biomarker is assessed mid-treatment (e.g. after 3 or 4 cycles of treatment) or post-treatment.

In another embodiment, the taxane and/or the anthracycline is administered in a chemotherapy regimen.

Another aspect includes a method of treating a subject afflicted with breast or ovarian cancer, the method comprising:
a. administering one or more doses of a taxane drug and/or anthracycline drug treatment to the subject;
b. scoring cancer cell toxicity/resistance and/or scoring clinical response in a subject afflicted with breast or ovarian cancer according to a method described herein; and
c. continuing the taxane drug and/or anthracycline drug treatment when the cancer cell is determined to be sensitive (e.g. the drug is toxic to the cell) and/or when the subject clinical response determined is a good clinical outcome or discontinuing the taxane drug and/or anthracycline drug treatment when the cancer cell is determined to be resistant and/or when the clinical response is determined to be a poor clinical outcome.

In another embodiment, the subject discontinues the taxane drug and/or anthracycline drug treatment when the score is indicative that the cancer cell is (and/or the cancer cell is identified as) resistant to the taxane drug and/or anthracycline drug treatment.

In yet another embodiment, the subject is treated with an agent selected from a TNFR1 agonist and a TNFR2 antagonist.

In an embodiment, the subject is also afflicted with a disease treatable by a TNF blocker and is being treated prior to administration of the one or more doses of the taxane drug and/or anthracycline drug with said TNFalpha blocker drug, wherein the subject discontinues the TNFalpha blocker prior to receiving the one or more doses of the taxane drug and/or anthracycline drug.

Another aspect includes use of a toxicity/resistance evaluation to treat a subject with breast or ovarian cancer, the use comprising evaluating toxicity resistance to a taxane drug and/or anthracycline drug in a cancer cell according to the method described herein and/or predicting clinical response in a subject afflicted with breast or ovarian cancer according to the method described herein, wherein the taxane drug and/or anthracycline drug treatment is to be continued when the cancer cell is determined to be responsive and/or when a good clinical outcome is predicted and the taxane drug and/or anthracycline drug treatment is to be discontinued when the cancer cell is determined to be resistant and/or when the clinical response is determined to be a poor clinical outcome.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the present disclosure will now be described in relation to the drawings in which:

FIG. 1: Docetaxel or paclitaxel-induced production of soluble tumor-necrosis factor alpha (sTNF-α) in MCF-7_{CC} and A2780 cells. Panel A depicts the mean concentration of sTNF-α (±standard error) (n=4) found in the medium of MCF-7_{CC} (black) or A2780 (white) cells cultured for 48 hours with varying amounts of docetaxel. Panel B depicts the mean concentration of sTNF-α (±standard error) (n=4) found in the medium of A2780 cells cultured for 48 hours with varying amounts of paclitaxel. Panel C depicts the effect of docetaxel (45 nM) and/or the nuclear factor (NF)-κB inhibitor SN-50 (7 μg/ml) on sTNF-α production in A2780 cells. The significance of differences in sTNF-α levels was assessed by using a Student t test; P values of <0.01 and <0.001 are represented by ** and *** symbols, respectively. It was also assessed whether another chemotherapy agent (doxorubicin) could, like the taxanes, induce TNFα expression in MCF-7_{CC} (panel D) and A2780_{CC} (panel E) cells.

FIG. 2: Acquisition of resistance to docetaxel or paclitaxel in MCF-7 cells. Sensitivity of MCF-7_{CC} cells (broken lines) and taxane-selected MCF-7 cells (solid lines) was measured after selection for survival to dose levels 8 (MCF-7_{TXT8}), 9 (MCF-7_{TXT9}), 10 (MCF-7_{TXT10}), 11 (MCF-7_{TXT11}), or 12 (MCF-7_{TXT12}), or at the maximally tolerated dose of paclitaxel (MCF-7_{TAX-1} cells). Mean survival fractions (±standard error) are plotted, and the significance of differences in docetaxel sensitivity between the taxane-selected and control cell lines was assessed by using a Student t test (n=5); P values of <0.05, <0.01, and <0.001 are represented by the *, **, and *** symbols, respectively.

FIG. 3: Sensitivity of MCF-7_{CC}, MCF-7_{TXT} and MCF-7_{TAX-1} cells to TNFα in clonogenic assays. The effect of docetaxel selection dose on colony formation in MCF-7_{CC} and MCF-7_{TXT} cells in the absence or presence of 10 ng/mL TNFα (panel A). Colony formation in MCF-7, MCF-7_{TXT-10} and MCF-7_{TAX-1} cells in the presence of 0 ng/mL, 10 ng/mL, 50 ng/mL or 100 ng/mL TNFα (panel B). The mean number of colonies (±standard error) are depicted, and significance of differences in TNFα sensitivity between MCF-7_{CC} and MCF-7_{TXT} cells at the various selection does was assessed using a Student's t-test; pvalues of <0.05, <0.01 and <0.001 are represented by the *, **, and *** symbols, respectively.

FIG. 4: Levels of soluble tumor-necrosis factor alpha (sTNF-α) in the medium of MCF-7_{CC} and MCF-7_{TXT} cells on exposure to docetaxel. The ability of MCF-7 cells to produce sTNF-α was measured by using an enzyme-linked immunosorbent assay (ELISA) after selection for survival to docetaxel dose levels 8 (MCF-7_{TXT8}), 9 (MCF-7_{TXT9}), 10 (MCF-7_{TXT10}), 11 (MCF-7_{TXT11}), or 12 (MCF-7_{TXT12}), or to the maximally tolerated dose of paclitaxel (MCF-7_{TAX-1} cells). After selection, the cells at the various selection doses were assessed for their production of sTNF-α in the absence (white bars) or presence (black bars) of 50 nM docetaxel is depicted in panel A. The results presented are the mean levels (±standard error) for five independent experiments, and the significance of differences in sTNF-α levels between MCF-7_{CC} and MCF-7_{TXT} cells was assessed by using a Student t test; P values of <0.01 and <0.001 are represented by the ** and *** symbols, respectively. Expression of TNF-α and S28 transcripts measured with RTqPCR by using cDNA preparations from MCF-7_{CC} and MCF-7_{TXT} cells (dose level 10) is depicted in panel B.

FIG. 5: Tumor-necrosis factor receptor (TNFR)1 and TNFR2 levels in MCF-7_{CC} and MCF-7_{TXT} cell lines. Panel A depicts protein levels of TNFR1, TNFR2, and GAPDH in MCF-7_{CC}, MCF-7_{TXT8}, MCF-7_{TXT9}, MCF-7_{TXT10}, and MCF-7_{TXT12} cells. Panel B depicts TNFR1 and S28 transcript levels in MCF-7_{CC} and MCF-7_{TxT10} cells determined with reverse transcription quantitative PCR (RTqPCR). Panel C depicts the mean concentration (±standard error; n=4) of secreted TNFR1 protein was examined in the cell lines by using an ELISA. Panel D depicts the ability of MCF-7_{CC} cells to form colonies in a clonogenic assay at various concentrations of docetaxel in the absence (broken line) or presence (solid line) of 5 μg/ml of a TNFR1 neutralizing antibody (R&D Systems). Error bars represent standard error of the mean. Significance of differences was assessed by using a Student t test; P values of <0.05, <0.01, and <0.001 are represented by *, **, and *** symbols, respectively.

FIG. 6: Networks of tumor-necrosis factor (TNF)-α-related genes that exhibited alterations in gene expression on selection for resistance to docetaxel in MCF-7 breast carcinoma (panel A), A2780 ovarian carcinoma (panel B), or MDA-MB-231 breast carcinoma (Panel C) cells. Gene expressions in the wild-type and docetaxel-resistant cell lines were compared with microarray analysis, after which differentially expressed genes were grouped into functional interaction networks. Genes upregulated in docetaxel-resistant cells are designated by a “+” symbol, whereas genes downregulated in docetaxel-resistant cells are depicted by a “−” symbol. Linker genes are depicted in diamonds. Direct activating or inhibitory interactions are indicated with the symbols → and ┤, respectively. Indirect interactions involving additional proteins are depicted with dashed lines.

FIG. 7: Taxane induced pathways expected to be associated with taxane cytotoxicity and taxane resistance. In taxane-sensitive cells a mechanism that may contribute to taxane sensitivity is postulated. The taxanes (boxed T symbol) diffuse across the plasma membrane where they activate the p65 (Rel A) and p50 (NF-κB1) subunits of the NF-κB dimer, which, binds to three known binding sites for NF-κB within the promoter region of the TNF gene. The increase in TNF transcription results in increased TNFα protein expression and, increased levels of sTNFα through the action of the protease ADAM-17. Binding of sTNFα preferentially to TNFR2 would promote further activation of NF-κB subunits and further production of TNFα and sTNFα in a positive feedback mechanism. The resultant high levels of sTNFα would also bind preferentially to TNFR1, resulting in formation of the death inducing signaling complex (DISC) and activation of caspase8-induced apoptosis in TNFR1-expressing cells. Continuous exposure to taxanes could result in selection against cells with active TNFR1 signaling pathways, resulting in taxane and TNF resistance. In taxane-resistant cells, TNFR1 signaling pathways are disrupted, for example by loss of TNFR1 expression/function. In cells lacking significant TNFR1 expression, the high levels of sTNFα induced by the taxanes would bind to TNFR2, resulting in activation of NF-κB and expression of NF-κB-dependent survival genes such as XIAP and Bcl2, without formation of the disk.

FIG. 8: Differences in the relative expression of the TNFα transcript between responders and non-responders at various times during treatment of MA.22 patients with epirubicin/docetaxel chemotherapy. Tumor RNA was isolated before, during, and after epirubicin/docetaxel chemotherapy in two patients that exhibited complete eradication of disease in the breast and axilla post-treatment (responders) and in two patients that showed substantial disease in the breast and axilla post-treatment (non-responders). Using reverse transcription quantitative PCR and gene-specific primers, the level of expression of the TNFα transcript in the RNA samples relative to the expression of seven housekeeping genes was quantified. The two responders exhibited almost a 20-fold increase in tumor TNFα transcript levels after treatment and the elevated levels persisted when a second RNA sample was isolated from the lesion post-treatment. In contrast, in the two non-responders, relative tumor TNFα expression was quite low throughout the treatment.

FIG. 9: Nuclear factor (NF)-κB activity in MCF-7_{CC}, MCF-7_{TXT}, and MCF-7_{TAX-1} cells. Panel A depicts levels of I-κB and a reference protein glyceraldehyde-3-phosphate dehydrogenase (GAPDH) in MCF-7_{CC}, MCF-7_{TXT8}, MCF-7_{TXT10}, and MCF-7_{TAX-1} cells in the absence or presence of 10 ng/ml Tumor-necrosis factor (TNF)-α. Protein levels were assessed in immunoblotting experiments with quantification of band intensities by using densitometry. The effect of selection for survival in increasing doses of docetaxel on the activity of the NF-κB p65 (panel B) and p50 (Panel C) subunits in the absence or presence of docetaxel also was examined by using an ELISA. Mean (±standard error) values are plotted, and the significance of differences between MCF-7_{CC} and taxane-resistant cells or differences between treated and untreated cells was assessed by using a Student t test; P values of <0.05, <0.01, and <0.001 are represented by *, **, and *** symbols, respectively.

FIG. 10: Effect of various agents on colony formation in MCF-7_{CC} or MCF-7_{TXT10} cells. MCF-7_{CC} or MCF-7_{TXT10} cells were assessed for their ability to form colonies after exposure to 10 ng/ml tumor-necrosis factor (TNF)-α, 10 μg/ml cycloheximide, or a combination of both agents for 24 hours as depicted in panel A. The ability of MCF-7_{TXT10} cells to form colonies in increasing concentrations of docetaxel in the absence (broken line) or presence (solid line) of a TNFR2 neutralizing antibody (5 μg/ml) was also examined as depicted in panel B. The effects of an NF-κB inhibitor SN-50 (7 μg/ml; broken gray line) or a control peptide SN-50 M (7 μg/ml; broken black line) on the colony-forming behavior of MCF-7_{TXT10} cells also were examined as depicted in panel C. Mean survival fractions (±standard error) are plotted. Significance of differences was assessed by using a Student t test; P values of <0.05, <0.01, and <0.001 are represented by *, **, and *** symbols, respectively.

FIG. 11: Reverse transcription quantitative PCR(RTqPCR) was used to assess differences in the expression of Tumor-necrosis factor (TNF-α)-related genes between MCF-7_{CC} and MCF-7_{TXT} cells (panel A) and between A2780 and A2780_{DXL} cells (panel B). For genes in which qPCR confirmed the changes in gene expression identified by cDNA microarray analysis, representative amplification plots are shown. S28 was used as the reference gene.

### DETAILED DESCRIPTION OF THE DISCLOSURE

### I. Definitions

The term “cancer” as used herein includes any cancer including breast cancer, ovarian cancer, a sarcoma, a lymphoma, various leukemias, uterine cancer, or lung cancer, as well as their subtypes and/or optionally taxane and/or anthracycline treatable cancers.

The term “breast cancer” as used herein includes all subtypes of breast cancer, including the HER2+, triple negative, basal, luminal A, luminal B, and “normal” subtypes of breast cancer as well as locally advanced breast cancer (LABC). Also included is invasive breast cancer, including but not limited to invasive breast cancer histologic types invasive ductal carcinoma, invasive lobular carcinoma, medullary carcinoma or tubular carcinoma. The breast cancer can be classified by grade including for example grade I, II or III. In another embodiment, the breast cancer is locally advanced breast cancer or inflammatory breast cancer. The breast cancer subtype can be also classified according to receptor status including hormone receptor positive, hormone receptor negative, her1+ve, her2−ve or functionally classified as luminal A, luminal b, basal*, TNBC, Her2+ve, or unclassified.

The term “ovarian cancer” as used herein means all subtypes of ovarian cancer, including the serous, clear cell, endometrioid, and mucinous subtypes.

The terms “patient” and “subject” which are used herein interchangeably refer to any member of the animal kingdom, preferably a human being including for example a subject that has or is suspected of having cancer, optionally breast cancer or ovarian cancer.

The term “biological sample” as used herein means any sample comprising cancer cells and/or pathologic tissue. For example, in embodiments where the sample is obtained from a subject, the pathologic tissue can comprise tumour and/or tumour microenvironment stromal tissue e.g. a biopsy of the remaining lesion. Post treatment for example, the biological sample can comprise tumour microenvironment stromal tissue. The biological sample can include for example, stromal rich areas of breast tumours including lymphatic vessels, blood vessels, and fibroblasts.

The term “cancer cell response” as defined herein refers to the extent of cell's cytotoxic response to the anti-proliferative or cytotoxic effects of an applied agent. These effects can include, but are not limited to, toxicity or sensitivity as indicated by cellular arrest, induced senescence and/or cell death or resistance (e.g. lack of cellular arrest, induced senescence and/or cell death).

The term “a cancer cell” as used herein means for example one or more cancer cells (e.g. tissue culture) or tissue comprising cancer cells including for example tumour tissue. Cancer cell and tumour cell are used herein interchangeably.

The term “changing cancer treatment” as used herein includes for example one or more of changing the dosage level, discontinuing the treatment, adding a chemotherapeutic agent(s) to the treatment or adding to or changing to an alternate cancer treatment such as radiation and/or surgery.

The term “decrease” or “increase” as used herein, means at least a 50% decrease or increase, at least a 60% decrease or increase, at least a 70% decrease or increase at least a 80% decrease or increase at least a 90% decrease or increase or at least a 1×, 2×, 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30× or greater decrease or increase compared to a control, optionally a selected reference threshold or pretreatment level.

The term “lack of increase” or “comparable to a reference threshold” as used herein in the context of a TNF biomarker level means less than a 40%, 30% or 20% change from a control. Alternatively, this change could be less than a 15% change, less than a 10% change, or less than a 10% change from a control such as a pretreatment control.

The term “control” as used herein refers to a suitable comparator biological sample or samples, such as the amount of TNF alpha produced by drug-sensitive tumour cells or tumours of that type in the absence of drug treatment and/or a subject pretreatment sample and/or a reference threshold derived therefrom. For example with reference to evaluating clinical outcome the control can be a suitable comparator biological sample from a subject or a group of individuals who are known as responders and/or a reference sample such as a pretreatment sample or earlier sample from the tested individual. For example, the control can be a biological sample obtained from a subject comprising breast cancer or ovarian cancer cell and/or tumour microenvironment. The control can also be a predetermined standard amount (e.g. associated with sensitivity or resistance) or reference threshold value e.g. “threshold associated with taxane and/or anthracycline resistance” determined from such suitable comparator samples.

The term “taxane drug” as used herein means a family of diterpenes that inhibit cell division by blocking microtubule depolymerization including but not limited to paclitaxel, docetaxel, larotaxel, Abraxane, docoxahexaenoic acid-linked paclitaxel, paclitaxel polyglumex, Ortataxel, Genexol, liposomal-encapsulated paclitaxel, and paclitaxel in a Vitamin E emulsion.

The term “anthracycline drug” as used herein means a family of red aromatic polyketide antibiotic chemotherapy drugs that act to prevent cell division by disrupting the structure and replication of DNA including but not limited to doxorubicin, epirubicin daunorubicin, idarubicin, valrubicin, and mitoxantrone in their wildtype or their pegylated forms.

The term “positive/good clinical response” as used herein refers to a positive therapeutic response to treatment, for example alleviation or amelioration of one or more symptoms or conditions, diminishment of extent of disease, stabilized (i.e. not worsening) state of disease, preventing spread of disease or preventing disease progression, delay or slowing of disease progression, reversal of disease, amelioration or palliation of the disease state, and remission (whether partial or total), whether detectable or undetectable. “A positive treatment outcome” can also mean prolonging survival and/or progression free survival as compared to expected survival if not receiving treatment, including for example an increased likelihood of a pathologic complete response (pCR) post-treatment, partial response and/or clinical response. In an embodiment, the positive/good clinical response is an increased likelihood to demonstrate a pCR post treatment.

The term “poor clinical response” to a lack of a therapeutic response to the treatment, for example no response (i.e. stable disease), recurrence of disease, or spread of disease (disease progression).

The term “responder” as used here means a cancer patient that demonstrates or is likely to demonstrate a positive treatment or therapeutic outcome, including for example, a measurable therapeutic response. Responders optionally include patients who demonstrate a pathological complete response (pCR) a partial response, or complete clinical response. In an embodiment, “responder” is a subject demonstrating and/or likely to demonstrate a pCR post treatment.

The term “non-responder” as used herein means a cancer patient that does not demonstrate or is not likely to exhibit a positive treatment outcome including for example no measurable therapeutic response, or for example a patient exhibiting a negative therapeutic outcome, such as progressive disease. An example includes a subject that exhibits extensive disease in the breast and axilla post treatment.

The term “resistant” as used herein refers to a cancer cell such as a breast cancer cell or an ovarian cancer cell or tumour response to a taxane and/or anthracycline, alone or in combination with another chemotherapeutic agent in a treatment regimen, where the cancer cells or subset of cancer cells within a tumour show no or insufficient response (for example the cells continue to grow) to the treatment in terms of cell death and/or post treatment outcome. In general, cancers that are resistant to chemotherapy will have clinical course similar to natural history without chemo; cancers susceptible to chemo will have clinical course better than natural history in terms of parameters such as time to recurrence/progression, 5 year survival, overall survival. Resistance for example can be relative to a parental cell or reference cell (e.g. relative to the average or median response of a group of cell samples or patients) and a fold resistance can be calculated. For tumour cell lines, this typically involves treating the wildtype and drug-resistant cell lines with various concentrations of a particular chemotherapy agent and determining in a clonogenic assay the concentration at which colony formation is reduced by 50% (IC₅₀). The resistance factor for the chemotherapy agent is then computed by dividing the IC₅₀ for the drug-resistant cell line by the IC₅₀ for the wildtype cell line.

The term “reference threshold” as used herein can be a cut-off value, above and/or below which a cancer cell type or tumour is identified as being resistant or sensitive/responsive to treatment and/or indicative of patient outcome derived from controls e.g. the amount of biomarker observed for tumours of that type in the absence of treatment or the pre-treatment value for a given patient. For example, a patient that has a TNF sensitivity biomarker level above a reference threshold is indicated to be responsive to the taxane and/or anthracycline treatment and/or predicts positive treatment outcome. The reference threshold can for example be derived from a control such as a pretreatment or untreated sample or a value derived from a population of subjects that are known responders and/or non-responders which for a preselected degree of specificity and sensitivity, classifies patients likely to be responders from patients likely to be non-responders.

As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, reversal of disease, amelioration or palliation of the disease state, and remission (whether partial or total), whether detectable or undetectable. “Treatment” can also mean prolonging survival as compared to expected survival if not receiving treatment.

The term “reference standard” as used herein with respect to measuring for example transcript and/or polypeptide level is a gene transcript or polypeptide standard that is more or less stable and hence useful for use as a reference standard for determining relative levels that is used for nomarlizing expression between samples and/or between a sample and control. As there can be variability in biopsy RNA stability after chemotherapy treatment and as typical house-keeping genes can also variable expression, at least 3, at least 4, at least 5, at least 6 or at least 7 reference genes are used for normalizing levels. Examples of reference genes whose transcripts could be used to normalize the expression level of TNFα-related transcripts include hydroxymethylbilane synthase (HMBS), hypoxanthine phosphoribosyltransferase 1 (HPRT1), mitochondrial ribosomal protein L19 (MRPL19), pumillo hornolog 1 (PUM1), ribosomal protein L13a (RPL13A), succinate dehydrogenase complex subunit A (SDHA), and splicing factor 3a, subunit 1 (SF3A1). The Genbank accession numbers for HMBS, HPRT1, MRPL19, PUM1, RPL13A, SDHA, and SF3A1 are M95623.1, BC000578.2, NM_{—}014763.3, AF315592.1, BC000514.2, NM_{—}004168.2, and BC007684.2 respectively and the amino acid and nucleotide sequences associated therewith are herein incorporated by reference.

The term “TNF biomarker” as defined herein refers to genes and gene products which are part of the TNFalpha, TNFR1 and TNFR2 signaling pathways, for example as documented in the Reactome™ knowledgebase (http://www.reactome.org/entitylevelview/PathwayBrowser.html#DB=gk current&FOCUS_S PECIES_ID=48887&FOCUS_PATHWAY_ID=109607&ID=83660), or as described in the review by Benn and Woolf (2004), Nature Reviews Neuroscience 5: 686-700.

It is demonstrated that TNF signaling pathway is associated with toxicity/resistance to chemotherapy agents. Several drug-resistant cell lines show changes in TNF-related pathways consistent with the activation of NF-κB survival pathways by TNF ligands.

Such biomarkers would also be expected to be differentially expressed and/or differentially activated in a taxane-resistant cancer cell compared with a taxane-responsive cancer cell (e.g. same taxane) and/or an anthracycline-resistant cancer cell compared with an anthracycline-responsive cancer cell (e.g. same anthracycline). In an embodiment, TNF biomarkers include TNFalpha, active NFkappaB, TNFR1 and TNFR2, the biomarkers listed in Table 2 and/or in FIG. 6. In an embodiment, the TNF biomarkers include TNFalpha, active NFkappaB, TNFR1, TNFR2, and/or the biomarkers listed in Table 2. In another embodiment, the TNF biomarkers include TNFalpha, active NFkappaB, TNFR1, TNFR2, BIRC3, TLR6 and TNFSF10. In yet another embodiment, the TNF biomarkers include TNFalpha, TNFR1, TNFR2 and active NFkappaB.

The term “Tumour necrosis factor-α” or “Tumor necrosis factor-alpha” (abbreviated herein as TNFalpha or TNFα), as used herein, refers to a cytokine, including for example Genbank Accession number NM_{—}000594.3, as well as naturally occurring variants, that in humans exists as a 17 kD secreted form (e.g. soluble TNF-alpha) and a 26 kD membrane associated form, the biologically active form of which is composed of a trimer of noncovalently bound 17 kD molecules. The structure of human TNFα is described further in, for example, Pennica, D., et al. (1984) Nature 312:724-729; Davis, J. M., et al. (1987) Biochemistry 26:1322-1326; and Jones, E. Y., et al. (1989) Nature 338:225-228. The term TNFα is intended to include recombinant TNFα which can be prepared by standard recombinant expression methods or purchased commercially (R & D Systems, Catalog No. 210-TA, Minneapolis, Minn.) as well as non-human TNFα.

The term “NFκB” or “NFkappaB” means nuclear factor activated kappa-light chain enhancer of activated B cells and is a protein complex of NFkappaB (including for example, p65, p50, p105, p100, c-Rel, etc) which when inactive is sequestered in the cytoplasm by a family of inhibitors (e.g IkappaBα, IkappaBβ) and when active is involved in regulating transcription of NFkappaB responsive genes.

The term “active NFkappaB” as defined herein means NFkappB that is capable of activating transcription, for example that has translocated to a cell's nucleus and which is not inhibited by IkappaB.

The term “TNF resistance biomarker” as defined herein means a TNF biomarker predominantly associated with cell survival, including for example TNFR2 pathway mediators including for example, TNFR2 and active NFkappaB, FIG. 6 TNFRII pathway mediators and/or genes listed in Table 2 that are identified as increased in expression in drug-resistant cells relative to control unselected cells.

The term “TNF sensitivity biomarker” as defined herein means as used herein a TNF biomarker predominantly associated with cell death, including for example TNFRI pathway mediators including for example TNFR1, FIG. 6 TNFRI pathway mediators and/or genes listed in Table 2 that are identified as having increased expression in drug-sensitive, control unselected cells, relative to its resistant counterpart (e.g. decreased expression in resistant cells).

The term “mid-treatment” as defined herein means after initiation of treatment for example after 2, 3 or 4 cycles of a 6 cycle treatment.

The term “post-treatment” as defined herein means after completion of a treatment regimen and/or an arm of a treatment regimen.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” Further, it is to be understood that “a,” “an,” and “the” include plural referents unless the content clearly dictates otherwise.

Further, the definitions and embodiments described in particular sections are intended to be applicable to other embodiments herein described for which they are suitable as would be understood by a person skilled in the art. For example, in the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### II. Methods

It is demonstrated herein that the taxane and anthracycline chemotherapy drugs induce tumour necrosis factor alpha (TNFα) production in breast and ovarian tumour cells that are responsive to taxane and anthracycline treatment.

In clinical samples, patients with non-responsive tumours to taxane and anthracycline treatment did not exhibit increases in TNFα during or post treatment whereas patients with responsive tumours exhibited increased TNFα during and post-treatment. For example, it is demonstrated herein that tumourTNFα transcript levels (relative to expression of a series of reference genes) were approximately 20-fold higher in patients that exhibited a pathologic complete response after docetaxel/epirubicin chemotherapy (responders), while TNFα transcript levels remained unchanged in patients with extensive disease in the breast and axilla post-treatment (nonresponders). This enhanced production of TNFα was also seen in the lesions of patients that exhibited a pCR post-treatment (unlike nonresponders). It is further shown herein that the chemotherapy drug doxorubicin can also induce TNFα production. TNFα and/or related genes appear able to serve as biomarkers of clinical response to taxanes (alone or in combination).

An aspect of disclosure provides a method of evaluating cancer cell drug response to a taxane drug and/or anthracycline drug in a cancer cell, the method comprising:
a. measuring a level of a TNF biomarker in a biological sample comprising the cancer cell after contacting the cancer cell with the taxane drug and/or anthracycline drug;
b. detecting a difference or a lack of difference in the level of the TNF biomarker compared to a control; and
c. scoring the cancer cell drug toxicity/resistance and/or identifying the cancer cell as sensitive or resistant to the to taxane drug and/or anthracycline drug according to the detected difference in the TNF biomarker level.

In an embodiment, the TNF biomarker is selected from TNFalpha, active NFkappaB, the biomarkers listed in Table 2 and/or FIG. 6.

In an embodiment, the TNF biomarker is selected from TNFSF13, TNFSF10, TLR6, TNFAIP3, TNFSF14, and BIRC3. In yet another embodiment, the TNF biomarker is selected from BIRC3, TLR6, and TNFSF10. It is demonstrated herein that these markers were increased in taxane resistant breast and/or ovarian cancer cell lines. For example BIRC3, TLR6, and TNFSF10, were also upregulated in resistant breast and ovarian cancer cells with TNFSF10 showing increased expression almost 300-fold in ovarian cancer taxane resistant cells.

In an embodiment, wherein the TNF biomarker to be measured is a TNF biomarker transcript, the measuring the TNF biomarker comprises preparing a RNA sample from the obtained biological sample, preparing a cDNA sample from the RNA sample, applying one or more primers to the cDNA sample that specifically bind to the TNF biomarker transcript making a primer TNF biomarker transcript complex, optionally applying one or more primers to the cDNA sample that specifically bind to one or more reference gene transcripts making a primer reference gene transcript complex, amplifying the TNF biomarker transcript and optionally the reference gene transcripts, applying a detection agent that detects the amplified TNF biomarker transcript and optionally the amplified reference gene transcript; and calculating the relative level of the TNF biomarker in the sample.

In an embodiment, the control is a pretreatment sample comprises tumour cells with low or average TNFalpha expression, e.g. low or average for that tumour type.

It is demonstrated that cells exposed to lower taxane selection doses (2-5 nM) can acquire resistance to taxanes when there is loss of the TNFR1 receptor. This allows the expressed TNF alpha to bind to TNFR2, which promotes NF-κB activation and activation of cell survival pathways.

In an embodiment, the TNFalpha level is determined in combination with TNFR level. In an embodiment, an increase of TNFalpha in the presence of TNFR1 expression is indicative of sensitivity. In another embodiment, an increase of TNFalpha in the presence of decreased TNFR1 is indicative of resistance. In yet another embodiment, an increase in TNFalpha in the presence of increased TNFR2 expression is indicative of resistance.

In an embodiment, chemotherapy-sensitive tumours produce increased levels of TNFalpha upon taxane and/or anthracycline treatment, express high levels TNFR1 pre-treatment and/or in response to taxanes, low levels of TNFR2 pre-treatment and/or upon treatment with taxanes and/or anthracyclines, and low levels of active NF-κB (prior to and/or upon treatment to taxanes and/or anthracyclines). The TNF biomarker levels are for example relative to reference standard gene expression levels.

In an embodiment, low relative TNF alpha transcript levels is as an expression ratio relative to the mean expression of the 7 reference genes (e.g. HMBS, HPRT1, MRPL19, PUM11, RPL13A, SDHA, and SF3A1) of 4.0 or less (during treatment) and 2.0 or less (post-treatment). For example as described below, responders at mid-treatment had relative TNF alpha expression ratios of 442 and 90, while the nonresponders had relative TNF alpha expression ratios of 4.0 and 0.8. Moreover, in the post-treatment data, responders at mid-treatment had relative TNF alpha expression ratios of 85 and 17, while the nonresponders had relative TNF alpha expression ratios of 1.1 and 0.35. Accordingly, in an embodiment, the control is a reference level corresponding to a low TNFalpha level. In an embodiment, the control is the pre-treatment TNF alpha transcript level (e.g. also normalized to the expression of the 7 reference genes). For example, as demonstrated below, responders mid-treatment exhibited a relative TNF transcript induction of 20.0 and 16.0 compared to pre treatment levels, while non-responders exhibited a relative TNF transcript induction of 0.66 and 0.85. Responders post-treatment exhibited a relative TNF transcript induction of 3.8 and 3.1, while non-responders exhibited a relative TNF transcript induction of 0.18 and 0.35. In an embodiment, a responder is a subject with a fold relative induction of 2 fold or greater (mid- or post-treatment), optionally with mid-treatment measures being preferable, since the effects of the drug on gene expression are likely higher while chemotherapy is being administered than after chemotherapy has been completed.

In an embodiment, high and low TNFR1 and TNFR2 levels are compared to levels seen in adjacent normal tissue.

In an embodiment, wherein the TNF biomarker to be measured is a TNF biomarker polypeptide, the measuring of the TNF biomarker comprises
a. applying an antibody specific for the TNF biomarker to the sample, wherein presence of the TNF biomarker creates an antibody-biomarker complex;
b. applying a detection agent that detects the antibody-biomarker complex by methods that include enzyme-linked immunosorbent assays (ELISAs), immunoblotting experiments, and/or immunohistochemical microscopy; and
c. scoring the cancer as responsive to the taxane and/or anthracyline drug and/or identifying the cancer cell as sensitive or resistant to the when the detection agent of step b) detects an increased level of a TNF sensitivity biomarker and/or a decreased level of a TNF resistance biomarker or as resistant to the taxane and/or anthracyline drug when the detection agent of step b) detects the absence of or a decreased level of a TNF sensitivity biomarker and/or an increased level of a TNF resistance biomarker.

The biological sample can for example be conjugated to solid surface for example as in a tissue array.

In another embodiment, the detecting a difference or lack of difference of the level of the TNF biomarker compared to the control comprises calculating the fold change and direction of change in the TNF biomarker level.

Scoring the cancer cell drug toxicity/resistance and/or identifying the cancer cell as sensitive or resistant to the to taxane drug and/or anthracycline drug according to the detected difference in the TNF biomarker level comprises in an embodiment, assessing if the TNF biomarker is a TNF resistant biomarker or a TNF sensitive biomarker, wherein relative increases in TNF sensitive biomarkers and relative decreases in TNF resistance biomarkers are assigned a positive value and relative decreases in TNF sensitive markers and relative increases in TNF resistance markers are assigned a negative value, positive scores are hence associated with sensitivity and negative scores are associated with resistance.

In an embodiment, the score is then a assigned to a zone that comprises subjects that have a range of scores which are more likely to be associated with a particular response, for example zone of 1 comprises scores likely to have a lack of response, zone 2 comprises scores likely to have an intermediate level of response, and zone 3 comprises scores likely to comprise strongly responding tumour. Reference thresholds or cut points that distinguish these zones can optionally be established based on established measures of clinical response, such as the complete irradiation of tumours post-treatment (pathologic complete response).

As discussed herein, the release of TNFα can for example promote the death of other tumour cells by binding to a receptor with a death effector domain (DED) called TNFR1. The release of TNF can also for example promote cell survival by binding a receptor lacking a DED called TNFR2. As demonstrated herein, cancer cells can evade taxane induced toxicity by modulating these pathways.

Accordingly, another aspect of the disclosure provides a method of evaluating cancer cell drug response to a taxane drug and/or anthracycline drug in a cancer cell, the method comprising:
a. measuring a level of TNFR1 activation and a level of TNFR2 activation in a biological sample comprising a cancer cell after contacting the cancer cell with the taxane drug and/or anthracycline drug;
b. detecting a difference or a lack of difference in the level of the TNFR1 activation and/or TNRF2 activation compared to a control; and
c. scoring the cancer cell drug toxicity/resistance and/or identifying the cancer cell as sensitive or resistant to the to taxane drug and/or anthracycline drug toxicity according to the detected difference in the TNFR1 compared to TNFR2 actviation.

A further aspect includes a method for predicting response in a patient by analyzing a subject sample for the presence or absence of a taxane and/or anthracycline drug sensitive cancer cell by measuring the level of one or more TNF biomarkers, wherein the subject is predicting to be responding to the taxane and/or anthracycline drug if an increased level of a TNF sensitive biomarker and/or a decreased level of a TNF resistance biomarker is detected.

In an embodiment, the method comprises contacting the cancer cell with taxane drug and/or anthracycline drug prior to measuring the level of the TNF biomarker and/or TNFR1 or TNFR2 pathway activation.

In an embodiment, the methods are used as a screening assay. In an embodiment, the method comprises:
a. contacting a cell population comprising a cancer cell with a test agent;
b. measuring a level of i) a TNF biomarker and/or ii) TNFR1 activation and TNFR2 activation in a sample of the test population comprising the cancer cell;
c. detecting a difference or a lack of difference in the level of the TNF biomarker and/or TNFR1 and TNFR2 activation compared to a control; and
d. scoring the cancer cell drug toxicity/resistance and/or identifying the cancer cell as sensitive or resistant to the to the test agent according to the detected difference in the TNF biomarker level and/or TNFR1 and TNFR2 activation level.

The test agent can for example be a taxane and/or anthracycline related compound or analog.

The increases and decreases and/or lack thereof are compared to a control. For example, an increased level of TNFα and/or activation of the TNFR1 pathway compared to a control is indicative of toxicity to the taxane drug and/or anthracycline drug and increased level of active NFκB and/or activation of TNFR2 pathway compared to a control is indicative of resistance to the taxane drug and/or anthracycline drug.

In an embodiment, the cancer or cancer cell is a cancer or cancer cell that is treatable by a taxane and/or an anthracycline and/or prodcues TNF in response to a taxane and/or an anthracycline.

Cancers that can be treated successfully with taxanes and anthracyclines include in addition to breast cancer and ovarian cancer, sarcoma, lymphoma, leukemias, uterine and lung cancers (Smith et al., BMC Cancer, 10:337) (Pharmacological Reviews June 2004 vol. 56 no. 2 185-229). The cancer can for example be any cancer that can produce TNF upon treatment with for example a taxane and/or an anthracycline.

In another embodiment, the cancer cell is a breast cancer cell or an ovarian cancer cell, a sarcoma cell, a lymphoma cell, a leukemic cell, a uterine cancer cell, colon cancer or a lung cancer cell. These can be from a cell line and/or obtained from a patient. For example, a biological sample comprising the cancer cell and/or pathologic tissue can be obtained in a cytological or histological biopsy. The biopsy can be for example a needle core biopsy or fine needle aspirate or a biopsy or resection obtained during surgery. For example, the breast cancer is locally advanced breast cancer (LABC), and/or the breast cancer is Her2, triple negative, basal subtype, luminal A, “normal” or luminal B subtype (e.g. luminal B1 or luminal b2) or optionally invasive ductal, invasive lobular or medullary cancer. In another embodiment, the ovarian cancer is epithelial, serous, mucinous, endometrioid, clear cell, or undifferentiated/unclassified ovarian cancer.

Increases and decreases of gene expression of TNF related genes which are associated with the acquisition of docetaxel resistance (survival/resistance biomarkers) or with promotion of docetaxel toxicity (sensitivity biomarkers) in MCF-7 breast tumor cells, MDA-MB231 breast tumor cells, and A2780 ovarian carcinoma cells are summarized in Table 2. In an embodiment, an increase in a level of a TNF resistance biomarker compared to a control and/or decrease in a level of a TNF sensitivity biomarker compared to a control is indicative of resistance and an increase in a level of a TNF sensitivity biomarker or a decrease in a level of a TNF resistance biomarker compared to the control is associated with toxicity to the taxane drug and/or anthracycline drug.

In another embodiment, an increase in active NFkappaB, and/or in a level of a biomarker listed as increased in Table 2 and/or a decrease in TNFR1 levels, and/or in a level of a biomarker listed as decreased in Table 2, compared to a control is indicative of resistance to the taxane drug and/or anthracycline drug.

In screening assay embodiments, an increase in a level of a TNF sensitivity biomarker, such as TNFalpha or a Table 2 biomarker identified as decreased, or a decrease in a level of a TNF resistance biomarker compared to the control is indicative of toxicity to the test agent and an increase in a TNF resistance biomarker such as an increase in active NFkappaB, and/or in a level of a biomarker listed as increased in Table 2 and/or a decrease in TNFR1 levels, and/or in a level of a biomarker listed as decreased in Table 2, compared to a control is indicative of resistance to and/or lack of efficacy of the test agent.

Increases in TNFalpha are shown herein to be associated with cytotoxicity in relation to taxane- and/or anthracycline responsive breast cancer cells and lack of increase is associated with resistance. Accordingly, in yet another embodiment, an increase in TNFalpha is indicative of toxicity/sensitivity to the taxane drug and/or anthracycline drug and/or the test agent.

In an embodiment, the level of the TNF biomarker, for example TNFα is increased at least 2×, 3×, 4×, 5×, 6×, 7×, 8×, 9×, 10×, 11×, 12×, 13×, 14×, 15×, 16×, 17×, 18×, 19×, or at least 20× compared to control. In another embodiment, the level of the TNF biomarker, is decreased at least 2×, 3×, 4×, 5×, 6×, 7×, 8×, 9×, 10×, 11×, 12×, 13×, 14×, 15×, 16×, 17×, 18×, 19×, or at least 20× compared to control. In another embodiment the level of the TNF biomarker is increased or decreased at least 21×, 22×, 23×, 24×, 25×, 26×, 27×, 28×, 29×, or at least 30× compared to a control.

In an embodiment, the control comprises a nonresistant cell or a resistant cell or is a standard amount and/or a reference threshold value associated with taxane and/or anthracycline resistance derived from one or more samples from non-responsive or responsive patients, pretreatment sample including for example an untreated cell or cell population.

In an embodiment, the control is a pre-treatment control, for example for quantifying the change in gene expression upon treatment (normalized to the expression of several reference genes). In an embodiment, the control is a reference threshold that is determined for a cancer type to distinguish between clinically responsive and clinically resistant tumours. In such an embodiment, the raw level of the biomarker as measured by various approaches or a level relative to the expression of various reference genes can be used, particularly if there is RNA degradation upon chemotherapy treatment.

In an embodiment, the control is a selected reference threshold value derived from a population of nonresistant cells and/or a population of resistant cells, wherein an increased level of a resistance TNF biomarker and/or a lack of increase or decreased level of a sensitive TNF biomarker are associated with taxane and/or anthracycline resistance or a decreased level of a resistance TNF biomarker and/or an increased level of a sensitive TNF biomarker are associated with taxane and/or anthracycline sensitivity.

In yet another embodiment, the TNF biomarker measured is TNF biomarker transcript. In another embodiment, the TNF biomarker measured is TNF biomarker polypeptide. In an embodiment, the TNF biomarker level measured is a relative amount.

In yet another embodiment, the TNFalpha measured is TNFalpha transcript. In a further embodiment, the TNFalpha measured is TNFalpha polypeptide. In an embodiment, the TNFalpha polypeptide measured is soluble TNFalpha polypeptide. Without wishing to be bound by theory, increased TNFalpha expression may leads to increased sTNFalpha production through the activity of for example the protease ADAM-17. Soluble TNFalpha binds preferentially to TNFR1 leading to for example caspase-8 induced apoptosis. In taxane-resistant cells, the TNFR1 pathway is disrupted and TNFalpha activates TNFR2, leading to activation of NFkappaB dependent survival genes.

In another embodiment, the TNF biomarker transcript (e.g. TNFalpha transcript) is measured using a method comprising polymerase chain reaction (PCR). In an embodiment, the PCR is quantitative PCR. In another embodiment, the PCR is RT-PCR.

In another embodiment, the TNF biomarker polypeptide, optionally, TNFalpha polypeptide is measured by enzyme linked immunosorbent assay (ELISA) or tissue microarray.

A skilled person would appreciate that there are numerous methods of quantifying polypeptide and/or transcript levels known in the art and the skilled person would readily recognize the appropriate reference standards suitable for each method. For example, as disclosed herein, transcript levels such as TNFalpha transcript levels can be determined by PCR. More preferably, transcript levels can be quantified using reverse transcriptase quanitative polymerase chain reaction (RTqPCR). Examples of primers suitable for RTqPCR of TNFalpha and other TNF-related genes are provided in Table 1. For RTqPCR of cells from a cell line, S28 is a suitable reference standard for normalizing expression readings. For RTqPCR of RNA extracted from cells of patients undergoing chemotherapy, reductions in RNA quantity and quality are common and multiple reference standards can be used. Suitable reference standards in these situations include for example HMBS, HPRT1, MRPL19, PUM1, RPL13A, SDHA, and SF3A1. Preferably, in situations involving an RNA source of reduced quality and/or quality RTqPCR expression results will be normalized against at least 2, at least 3, at least 4, at least 5, at least 6, or at least 7 reference standards.

In an embodiment, the level of the TNF biomarker is measured using primers listed in Table 1.

In an embodiment, the level of TNFalpha transcript is measured using a primer set comprising 5′-TCTTCTCGAACCCCGAGTGA-3′, Reverse: 5′-GGAGCTGCCCCT-CAGCTT-3′.

In another embodiment, the level of TNFR1 transcript is measured using a primer comprising 5′-ACTGCCTCAGCTGCTCCAAAT-3′ and/or 5′-CCGGTCCACTGTGCAAGAA-3′.

Methods of quantifying proteins, like sTNFα and sTNFR1 (e.g. soluble TNFα and soluble TNFR1), and cellular proteins from soluble extracts include quantification by ELISA. For example, sTNFalpha was quantified using ELISA kits from R&D Systems™ following the manufacturer's instructions. Immunohistochemistry can also be used. Similarly, active NFkappaB can be quantified from nuclear extracts using a TransAM™ NF-κB Family ELISA kit (Active Motif™, Carlsbad, Calif.). Other expression analysis methods disclosed herein include methods common in the art, including immunoblotting and microarray analysis.

Another aspect includes a method of evaluating cancer cell drug resistance to a taxane drug and/or anthracycline drug, the method comprising:
a. measuring a level of a TNF biomarker in a biological sample comprising the cancer cell after contacting the cancer cell with the taxane drug and/or anthracycline drug;
b. detecting a difference or lack of difference in the relative level of the TNF biomarker compared to a control; and
c. scoring the clinical response and/or predicting the clinical response for the subject according to the detected difference in the relative level of the TNF biomarker;

wherein a difference in the level of the TNF biomarker compared to the control (e.g. resistant cell, a non-resistant cell, standard amount and/or a reference threshold associated with taxane and/or anthracycline resistance/toxicity) is indicative of resistance of the cancer cell to the taxane drug and/or anthracycline drug.

In an embodiment, the difference is between pre-treatment and mid-treatment samples. In another embodiment, the difference is between clinically responsive and clinically resistant tumours (at either the pre-treatment or mid-treatment time points).

Interestingly, it was observed that MCF-7 breast tumour cells selected for resistance to paclitaxel (MCF-7TAX-1) or docetaxel (MCF-7TXT) exhibit resistance to TNFα, with the latter cell line producing 200-fold more TNFα than its drug-sensitive parent at intermediate selection doses and no TNFα at high selection doses. Without wishing to be bound to theory but since prolonged exposure to taxanes selected in some instances for cells deficient in TNFR1 levels, high levels of secreted TNFα could bind to a receptor lacking a DED (e.g. TNFR2), promoting NF-κB-induced expression of pro-survival genes. Alternatively, the expression of drug efflux pumps may prevent taxanes from accumulating sufficiently well into tumour cells preventing activation of taxane-induced TNF production. Similarly, reduced tumour vascularization may also prevent sufficient taxane uptake into tumours to promote taxane-stimulated activation of TNF production.

In yet another embodiment, the cancer cell is a primary cancer cell. In yet another embodiment, the primary cancer cell is contacted with the taxane drug and/or anthracycline drug in vitro. In another embodiment, the primary cancer cell is contacted in vivo, e.g. by administering to a subject in need thereof a taxane drug and/or anthracycline drug.

Accordingly, another aspect provides a method of evaluating cancer cell response to administration of a taxane drug and/or anthracycline drug in a subject in need thereof, the method comprising:
a. measuring a level of a TNF biomarker in a biological sample comprising pathologic tissue obtained from the subject after administering to the subject of one or more doses of the taxane drug and/or anthracycline drug; and
b. detecting a difference or lack of difference in the level of the TNF biomarker compared to a control; and
c. scoring the cancer cell drug toxicity/resistance and/or identifying the cancer cell as sensitive or resistant to the to taxane drug and/or anthracycline drug toxicity according to the detected difference in in the TNF biomarker level.

In an embodiment, the method further comprises administering one or more doses of the taxane drug and/or anthracycline drug to the subject prior to measuring the level of a TNF biomarker in the biological sample comprising pathologic tissue.

In an embodiment, the TNF biomarker is TNFα. As described below, increases in TNFα are associated with good clinical outcome.

Accordingly further aspect provides a method of evaluating clinical response in a subject afflicted with breast cancer or ovarian cancer, the method comprising:
a. measuring a level of a TNF biomarker, optionally TNFα, in a biological sample comprising pathologic tissue obtained from the subject after administering to the subject one or more doses of the taxane drug and/or anthracycline drug;
b. detecting a difference or lack of difference in the level of the TNF biomarker compared to a control; and
c. scoring the clinical response and/or predicting the clinical response for the subject according to the detected difference in in the relative leve of the TNF biomarker;
   wherein i) an increase in a level of TNFα and/or a TNF sensitivity biomarker is indicative that the subject is positively responding and/or will have a positive clinical response or ii) a lack of increase or a decrease in a level of TNFα and/or a TNF sensitivity biomarker and/or an increase in a level of a TNF resistance biomarker is indicative that the subject is negatively responding and/or will have a poor clinical response.

In an embodiment, the clinical response is progressive disease, stable disease, partial response, complete clinical response or pathological complete response.

In another embodiment, an increase in a TNFα level is predictive of a good clinical response.

In an embodiment, the method comprises:
a. measuring a level of a TNFα in a biological sample comprising pathological tissue obtained from the subject after administration of the one or more doses of the taxane drug and/or anthracycline drug; and
b. detecting a difference in the level of the TNFα compared to a control;
c. optionally scoring the cancer cell drug toxicity/resistance and/or identifying the cancer cell as sensitive or resistant to the to taxane drug and/or anthracycline drug toxicity according to the detected difference in in the TNF biomarker level;
   wherein i) an increase in the level of TNFα is indicative that the subject is positively responding and/or will have a positive clinical response or ii) a lack of increase or decrease in the level of the TNFα is indicative that the subject is negatively responding and/or will have a poor clinical response.

In an embodiment, the method further comprises:
a. administering one or more doses of a taxane drug and/or anthracycline drug to the subject prior to measuring the level of the TNFα in the biological sample comprising pathological tissue obtained from the subject

In an embodiment, the method comprises detecting an increase in the level of TNFα in a biological sample comprising pathological tissue compared to a control. In an embodiment, the level of TNFα is a relative level, relative to 1, 2, 3 4, 5, 6, 7 or more reference standard transcripts or polypeptide levels. For example, in an embodiment, a relative amount of TNFα standardized to a plurality of reference standards is compared to a relative level of TNFα in a control.

In an embodiment, the expression level of each reference gene is measured by quantitative pCR (e.g. fluorescent units minus background); a mean level (+/−SE) is calculated for all of the reference genes; the mean+/−SE of the TNF-related biomarker level is divided by the mean+/−SE for the reference genes, providing a relative expression level. In an embodiment, a reference threshold is then determined for each biomarker that best differentiates between clinically responsive and clinically resistant tumours. In an embodiment, the sample is assigned a zone, optionally zone 1 and 2, wherein subjects are characterized as likely not responding or likely responding respectively or zones 1, 2 and 3, wherein subjects are characterized as likely not responding, likely having an intermediate response (or comprise indeterminate subjects) or likely responding respectively. In an embodiments with multiple biomarkers, a mean score can be calculated.

In one embodiment, the drug is administered systemically.

In another embodiment, the drug is administered directly to the tumour.

In another embodiment, an increase in a TNF resistance biomarker compared to the control and/or lack of increase or decrease in a TNF sensitivity biomarker compared to the control predicts poor clinical response.

In an embodiment, biological sample is obtained mid treatment. In another embodiment, the sample is obtained post treatment.

In another embodiment, the taxane drug is selected from paclitaxel, docetaxel, larotaxel, Abraxane, docoxahexaenoic acid-linked paclitaxel, paclitaxel polyglumex, Ortataxel, Genexol, liposomal-encapsulated paclitaxel, and paclitaxel in a Vitamin E emulsion. Any taxane drug or taxane comprising regimen can be used.

In an embodiment, the level of TNF biomarker is a intracellular level of TNF biomarker. In another embodiment, the level of TNF biomarker is a tissue level of TNF biomarker.

In another embodiment, the anthracycline drug is selected from epirubicin, doxorubicin, epirubicin, daunorubicin, idarubicin, valrubicin, and mitoxantrone. Any anthracycline and/or anthracycline comprising regimen can be used.

In another embodiment, the taxane and/or the anthracycline is administered in a chemotherapy regimen.

Another aspect includes a method of treating a subject afflicted with breast or ovarian cancer, the method comprising:
a. administering one or more doses of a taxane drug and/or anthracycline drug treatment to the subject;
b. evaluating cancer cell toxicity/resistance and/or evaluating clinical response in a subject afflicted with breast or ovarian cancer according to a method described herein; and
c. continuing the taxane drug and/or anthracycline drug treatment when the cancer cell is determined to be responsive and/or when the clinical response determined is a good clinical outcome or discontinuing the taxane drug and/or anthracycline drug treatment when the cancer cell is determined to be resistant and/or when the clinical response is determined to be a poor clinical outcome.

In another embodiment, the subject discontinues the taxane drug and/or anthracycline drug treatment.

In an embodiment, the subject is also afflicted with a disease treatable by a TNF inhibitor drug and is being treated prior to administration of the one or more doses of the taxane drug and/or anthracycline drug with said TNFalpha inhibitor drug, wherein the subject discontinues the TNFalpha blocker prior to receiving the one or more doses of the taxane drug and/or anthracycline drug.

Examples of TNFalpha inhibitor drugs include TNF antagonists such as TNFalpha monoclonal antibody such as infliximab (Remicade), adalimumab (Humira), certolizumab pegol (Cimzia), and golimumab (Simponi), circulating TNF receptor fusion proteins such as etanercept (Enbrel) as well as small molecule TNFalpha inhibitors.

Demonstrated herein is use of selective TNFR2 antagonists which can increase responsiveness in taxane resistant breast and ovarian cancer cells.

A further aspect includes a method of increasing sensitivity of a taxane and/or anthracycline resistant cancer cell to a taxane and/or anthracycline drug, the method comprising:
a. contacting the cancer cell with a selective TNFR1 agonist and/or a selective TNFR2 antagonist in combination with the taxane drug and/or the anthracycline drug.

In embodiments where the cell is in vivo, the method comprises:
a. administering to a subject in need thereof a selective TNFR1 agonist and/or a selective TNFR2 antagonist in combination with one or more doses of a taxane drug and/or an anthracycline drug.

A selective TNFR1 agonist is for example an agent that stimulates TNFR1 signaling but has little or no effect on TNFR2 signaling. Similarly, a selective TNFR2 agonist is for example a molecule that stimulates TNFR2 signaling but has little or no effect on TNFR1 signaling. Selective TNFR1 agonists include for example TNF polypeptides which bind preferentially to TNFR1 relative to TNFR2 or antibodies or fragments thereof that specifically bind to TNFR1 and stimulate TNFR1 signaling. A selective TNFR2 antagonist is a molecule which for example preferentially inhibits the binding of TNFα to TNFR2 relative to TNFR1 and include for example antibodies or fragments thereof which bind TNFR2 but do not bind TNFR1. Methods for producing suitable antibodies are known in the art.

In an embodiment, the TNFR1 agonists and/or TNFR2 antagonists are those described in US20100034808 and/or EP1875247A2, incorporated herein by reference in their entirety.

The taxane and/or anthracycline can be administered contemporaneously with the selective TNFR1 agonist and/or a selective TNFR2 antagonist, and/or subsequent to administering the selective TNFR1 agonist and/or a selective TNFR2 antagonist.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

### National Clinical Trial (NCIC-CTG-MA.22)

To evaluate whether the TNF pathway has a role in clinical response to taxanes and/or anthracyclines, tumour biopsies were taken from 93 locally advanced breast cancer patients who were enrolled in the NCIC-CTG MA.22 clinical trial. Six core biopsies were collected from these patients prior to, during (after 3 or 4 cycles), and post epirubicin/docetaxel combination chemotherapy (after 6 or 8 cycles), depending upon the dosing regimen. Three cores were retained for immunohistochemical receptor expression studies, while the remaining three cores were flash frozen in liquid nitrogen. RNA was isolated from 2 or 3 of the flash frozen biopsies from each patient at the three time points and gene profiling conducted on samples of sufficiently high RNA quality (RIN≧5.0) (details at www.ClinicalTrials.gov (ClinicalTrials.gov Identifier: NCT000002866) (49).

Using a RTqPCR approach, tumour TNFα transcript levels before, during, and after epirubicin/docetaxel chemotherapy were compared between two patients that exhibited a pCR post-treatment (responders) and two patients that retained extensive disease in the breast and axilla post-chemotherapy (non-responders). As shown in FIG. 8, TNFα expression in responders (relative to that of 7 reference genes increased ˜20-fold over pre-treatment levels after 3 to 4 cycles of chemotherapy and this increase persisted (at a ˜10-fold level) in the post-treatment lesions. In contrast, in non-responders, relative TNFα expression did not change appreciably during and post-treatment. These findings are compelling because they appeared consistent within each patient, and with multiple independent biopsies and RNA isolations throughout the course of therapy.

These findings suggest that TNF alpha transcript levels are very low in these tumours. It is only upon treatment with docetaxel/epirubicin that TNF alpha transcript expression is increased (and only in chemoresponsive tumours) that likely accumulate sufficient docetaxel to promote TNF alpha transcript expression

In vitro data shows that doxorubicin induces TNFα expression in MCF-7 breast and A2780 ovarian tumour cells (FIGS. 1D & 1E). This study also indicates that there appears to be little need for concern of variations in tissue sampling for a given tumour during treatment, as duplicate core biopsies taken from each patient at the various treatment times showed very similar relative TNFα expression.

Pre-treatment levels of TNFα transcript were not significantly different between responding and non-responding tumours. It should also be noted that the two responding patients had basal tumour subtypes, while the two non-responding patients had basal and luminal tumours. This suggests that tumour TNFα transcript levels during treatment were better indicators of toxicity than pre-treatment tumour subtyping. The responders were administered the standard dose regimen, and the non-responders were administered the dose dense regimen (there were pCRs achieved in patients given the dose dense regimen).

### Example 2

### Methods

### Cell Culture and Maintenance

MCF-7 cells from the American Tissue Culture Collection (catalog number HTB-22) were cultured or selected for survival in increasing doses of docetaxel or paclitaxel as previously described [17;18]. The initial concentrations of docetaxel and paclitaxel used to begin selection (dose 1) were 0.51 and 0.56 nM, respectively. Cells selected to docetaxel concentrations of 1.11 nM (dose 8, MCF-7_{TXT8}), 3.33 nM (dose 9, MCF-7_{TXT9}), 5.00 nM (dose 10, MCF-7_{TXT10}), 15 nM (dose 11, MCF-7_{TXT11}), and 45 nM (dose 12, MCF-7_{TXT12}) were used in this study. Numbers in subscripts of cell line names refer to the maximum docetaxel dose level to which the cells were exposed. The paclitaxel resistant cell line used in this study was selected in an identical manner to a final concentration of 6.64 nM paclitaxel (MCF-7_{TAX-1} cells; hyphenated number indicates the first cell line selection, not drug dose). MCF-7 cells were also “selected” in the absence of taxanes to passage numbers similar to those of drug-selected cells to control for genotypic or phenotypic changes associated with long term culture (“co-cultured control” MCF-7_{CC} cells). A2780 ovarian carcinoma cells from the European Collection of Cell Cultures were also selected for resistance to docetaxel in an identical manner (A2780_{DXL} cells), including the creation of “co-cultured control” A2780_{CC} cells.

Measurement of sTNFα and sTNFR1 in Cell Culture Media

Concentrated proteins from the medium of two million MCF-7_{CC}, MCF-7_{TXT} or A2780 cells (grown in culture in the absence or presence of various concentrations of paclitaxel or docetaxel) were assessed for levels of sTNFα or sTNFR1 using ELISA kits from R&D Systems™, following the manufacturer's instructions.

### Clonogenic Assays

Cellular sensitivity to TNFα or docetaxel was assessed using a clonogenic assay as described previously [17]. Docetaxel resistance factors for the cell lines were determined by dividing the IC₅₀ for docetaxel in the taxane-resistant cell lines by the IC₅₀ for MCF-7_{CC} cells. In some experiments, cells were exposed to 1 μg/ml cycloheximide, TNFR1 or TNFR2 neutralizing antibodies from R&D Systems™ (both at 5 μg/ml), or a peptide from Calbiochem Laboratories™ (La Jolla, Calif.), which potently blocks NF-κB function by inhibiting translocation of the NF-κB complex into the nucleus [60] (SN-50, 7 μg/ml). A control peptide at the same concentration (SN-50M) was used in the latter experiments to assess the specificity of NF-κB inhibition.

### Immunoblotting Analysis

MCF-7_{CC}, MCF-7_{TXT}, and MCF-7_{TAX-1} cells were incubated in the absence or presence of 20 ng/mL TNFα for 24 hours. Cells were extracted in RIPA buffer and 100 μg of extract proteins assessed for the expression of specific proteins using standard immunoblotting procedures as previously described [17]. Antibodies used in these experiments included TNFR1-, TNFR2- and IκB-specific antibodies from Cell Signaling Technology™ (Danvers, Mass.) and a mouse-derived glyceraldehyde 3-phosphate dehydrogenase (GAPDH) antibody from Santa Cruz Laboratories™. Densitometric quantitation of bands generated by the IkB antibody was performed using AlphaEaseFC™ software (Alpha Innotech™, San leandro, CA). Band intensity was normalized relative to GAPDH band intensity.

### Quantification of TNFR1 and TNFα Transcript Levels by RTQPCR

The levels of TNFR1 and TNFα transcripts in MCF-7_{CC} and MCF-7_{TXT10} cells were assessed as described previously [98] using the following primers: TNFR1: Forward: 5′-ACTGCCTCAGCTGCTCCAAAT-3′ (SEQ ID NO:1), Reverse: 5′-CCGGTCCACTGTGCAAGAA-3′ (SEQ ID NO:2), TNFα: Forward: 5′-TCTTCTCGAACCCCGAGTGA-3′ (SEQ ID NO:3), Reverse: 5′-GGAGCTGCCCCT-CAGCTT-3′, (SEQ ID NO:4) S28: Forward: 5′-TCCATCATCCGCAATGTAAAAG-3′ (SEQ ID NO:5), Reverse: 5′-GCTTCTGCGTCTGACTCCAAA-3′ (SEQ ID NO:6).

cDNAs were cDNA was placed into each well and gene-specific primers (300 nM) were added. Reactions were diluted 1:2 with SYBR Green I Master Mix (Applied Biosystems), and amplification by PCR was performed as follows: 1 cycle of 95° C. for 10 min and (40 cycles of 95° C. for 15 s, 55° C. for 15 s, and 72° C. for 30 s), representing the melting, primer annealing, and primer extension phases of the reaction, respectively. Following the amplification, a reaction product melt curve was performed to provide evidence for a single reaction product.

### Measurement of NF-κB Activity

MCF-7_{CC} and MCF-7_{TXT} cells were cultured in the presence or absence of 50 nM docetaxel for 24 hours. The activity of the NF-κB p65 and p50 subunits in 10 μg of nuclear extracts was assessed as outlined in the TransAM™ NF-κB Family ELISA kit (Active Motif™, Carlsbad, Calif.). Readings at 450 nm were normalized to the sum of all readings on the plate in order to compare across triplicate experiments.

Identification of Changes in Gene Expression Associated with the Acquisition of Docetaxel Resistance

Agilent™ 4×44k human genome oligonucleotide arrays were used to profile differences in gene expression between MCF-7_{TXT} and MCF-7_{CC} cells at selection dose 10 and between docetaxel-resistant and wildtype A2780 ovarian carcinoma cells (A2780_{DXL} and A2780 cells, respectively) at the maximally tolerated dose using MIAME standards [99]. RNA was isolated from each cell line using RNeasy™ Mini kits (Qiagen™, Mississauga, ON) and 500 ng of each RNA preparation was labelled and amplified using Agilent™ Quick Amp labeling kits. The labelling and array hybridization procedures were performed as per the manufacturer's protocol for a 2 color microarray experiment.

Identification of Differences in Gene Expression Associated with Docetaxel Resistance

The hybridized microarrays were scanned using Agilent™ scanners and feature extraction software (version 10_{—}7_{—}3_{—}1) and differentially expressed genes associated with the acquisition of docetaxel resistance identified using Partek™ Genomic Suite (Partek, Inc., St. Louis, Mo.). The background-corrected intensity values were used for analysis. A 3-way ANOVA was performed to identify significant changes in gene expression using the Method of Moments [100]. Genes with >2-fold differences in gene expression were selected with a false discovery rate of either 0.05 or 0.01 [101]. The data from these array experiments have been deposited in the National Centre for Biotechnology Information Gene Expression Omnibus database (accession number GSE26129) at the following url: http://www.ncbi.nlm.nih.gov/geo/query/acc.cgi?token=hrkztqqskcgsmpu&acc=GSE26129.

### Network-based Analysis of Gene Expression

In order to determine whether the above-described changes in gene expression associated with acquisition of docetaxel resistance in breast or ovarian tumour cells may reflect changes in the function of specific biochemical pathways in these cells, the genes identified as being associated with docetaxel resistance were subjected to functional interaction (FI) network analysis [46]. Briefly, the FI network covers ˜50% of the human proteome representing >200,000 functional interactions. Pairwise shortest paths among genes of interest in the FI network were calculated and hierarchically clustered (based on the average linkage method). Clusters were then selected containing more than 90% of altered genes. To calculate a p-value for the average shortest path, a 1000-fold permutation test was performed by randomly selecting the same number of genes from the biggest connected network component. A minimum spanning tree algorithm was used to find linkers that connected all genes of interest in one subnetwork [102]. The Markov Cluster Algorithm (MCL) was used [103] with inflation of 1.6 for network clustering. Only the biggest clusters with numbers of proteins not <2% of the total network were taken into account. All network diagrams were drawn using Cytoscape™ [104]. The functional enrichment analysis for pathways was based on a binominal test. A false discovery rate was calculated based on 1000 permutations on all genes in the FI network. This network-based analysis was also applied to another dataset that documents differences in gene expression between docetaxel-resistant and parental MDA-MB-231 breast cancer cell lines (Gene Expression Omnibus (GEO) accession number GSE28784).

### Confirmation of Microarray-based Changes in Gene Expression by Quantitative Reverse Transcription Quantitative Polymerase Chain Reaction (RTqPCR)

A number of the TNFα-related genes in the above networks were further assessed for expression in wildtype and docetaxel resistant MCF-7 and A2780 cells by RTqPCR using our above-described method and primers depicted in Table 1.

| | Forward Primer | Reverse Primer | SEQ ID |
|---|---|---|---|
| S28 | 5′TCC ATC ATC CGC AAT GTA AAG-3′ | 5′-GCT TCT CGC TCT GAC TCC AAA-3′ | 5, 6 |
| TNFAIP3 | 5′-GAC CAT GGC ACA ACT CAT | 5′-GTT AGC TTC ATC CAA CTT TGC GGC | 7, 8 |
| | CTC A-3′ | ATT G-3′ | |
| TNFSF10 | 5′-CGT GTA CTT TAC CAA CGA GCT GA-3′ | 5′-ACG GAG TTG CCA CTT GAC TTG-3′ | 9, 10 |
| TNFSF13 | 5′-ACT CTC AGT TGC CCT CTG GTT G-3′ | 5′-GGA ACT CTG CTC CGG GAG ACT C-3′ | 11, 12 |
| TNFSF14 | 5′-TTT GCT CCA CAG TTG GCC TAA TC-3′ | 5′-CAA TGA CTG TGG CCT CAC CTT C-3′ | 13, 14 |
| TLR1 | 5′-GGT ACC AGG CCC TCT TCC TCG | 5′-TAG GAA CGT GGA TGA GAC CG | 15, 16 |
| | TTA G-3′ | TTT TT-3′ | |
| TLR6 | 5′-GCA AAA ACC CTT CAC CTT GTT | 5′-CCA AGT CGT TTC TAT GTG GTT | 17, 18 |
| | TTT C-3′ | GAG G-3′ | |
| BIRC3 | 5′-TGT TGG GAA TCT GGA GAT GA-3′ | 5′-CGG ATG AAC TCC TGT CCT TT-3′ | 19, 20 |
| TNFR1 | 5′-ACT GCC TCA GCT GCT CCA AAT-3′ | 5′-CCG GTC CAC TGT GCA AGA A-3′ | 1, 2 |
| TNFα | 5′-TCT TCT CGA ACC CCG AGT GA-3′ | 5′-GGA GCT GCC CCT CAG CTT-3′ | 3, 4 |

Table 1 primers selected for confirmation of changes in the expression of TNFα-related genes by reverse transcription quantitative polymerase chain reaction (RTqPCR). Reaction conditions were as described above and expression was assessed relative to that of S28, the internal reference gene.

Assessment of Relative TNF Alpha Expression in MA.22 Patient Biopsies Before, During, and after Epirubiicn/Docetaxel Chemotherapy by High Throughput RTqPCR

RNA reverse transcription was performed as previously described by Brosseau et al. [RNA (2010) 16: 442-449] using 50-250 ng_of RNA from MA.22 patient biospies with “Transcriptor,” random primer p(dN)₆, dNTPs_(Roche Diagnostics), and RNase OUT (Invitrogen). Total cDNA_was further purified on a QIAquick_PCR purification kit (Qiagen). Total cDNA was pooled and_diluted to 0.33-3.33 ng/mL. All forward and reverse primers were_individually resuspended at 20-100 mM stock solution in Tris-EDTA buffer (IDT) and diluted as a primer pair to 1 mM in RNase_DNase-free water (IDT). Ten microliters of real-time PCR reactions were performed either in a 7500 ABI apparatus (Applied_Biosystems) or a Realplex (Eppendorf) with 5 mL of FastStart_Universal SYBR Green Master mix (Roche Diagnostics) with the following cycling: 10 min at 95° C.; 50 cycles: 15 sec at 95° C., 30 sec_at 60° C., 30 sec at 72° C.; and melting curve: 15 sec at 95° C., 60 sec_at 60° C.; 1° C./min temperature gradient, 15 sec at 95° C., using 3 mL_(totaling 1-10 ng) of a template cDNA or diluted amplicons in_tRNA (50 ng/mL; Ambion) and 2 mL of a 1 mM solution offorward and reverse primer. TNFα transcript levels were quantified relative to the expression of reference genes HMBS, HPRT1, MRPL19, PUM1, RPL13A, SDHA, and SF3A1 using the following primers:

TABLE 3
Reference standard primers

| Gene | Fwd seq | Rev seq | SEQ ID NOs |
|---|---|---|---|
| HMBS | GCTTCACCATCGGAGCCATCT | TGGCAGGGTTTCTAGGGTCTT | 21, 22 |
| HPRT1 | TGACACTGGCAAAACAATGCA | GGTCCTTTTCACCAGCAAGCT | 23, 24 |
| MRPL19 | AAGGAGAAAAGTACTCCACATTCCAGAG | TGGGTCAGCTGTAGTAACACGA | 25, 26 |
| PUM1 | TGAGGTGTGCACCATGAAC | CAGAATGTGCTTGCCATAGG | 27, 28 |
| RPL13A | CCTGGAGGAGAAGAGGAAAGAGA | TTGAGGACCTCTGTGTATTTGTCAA | 29, 30 |
| SDHA | TGTTGATGGGAACAAGAGGGCA | GCCTACCACCACTGCATCAAAT | 31, 32 |
| SF3A1 | GGAGGATTCTGCACCTTCTAA | GCGGTAGTAGGCATGGTAA | 33, 34 |
| TNF | TGCCTGCTGCACTTTGGAGT | TGGGCCAGAGGGCTGATTAGA | 35, 36 |

### Results

### A Novel Role for TNFα in Taxane Cytotoxicity and Resistance:

While taxanes are well known to induce mitotic arrest through microtubule stabilization, it is demonstrated herein and reported [44] that these drugs can substantially elevate tumour necrosis factor alpha (TNFα) production in breast (MCF-7) and ovarian (A2780) tumour cells at clinically relevant concentrations 3 nM) (FIG. 1). In this study, MCF-7 breast tumour cells were also selected for survival in increasing concentrations of paclitaxel or docetaxel (MCF-7_{TAX-1} and MCF-7_{TXT} cells, respectively). As a control, MCF-7 cells were selected in an identical manner in the absence of drug (MCF-7_{CC} cells). It was observed that MCF-7_{TAX-1} and MCF-7_{TXT} cells acquired resistance to paclitaxel and docetaxel, respectively, when the drug selection dose reached or exceeded a particular level (dose 9, FIG. 2). Interesting, both cell lines also acquired substantial resistance to TNFα (FIG. 3), while MCF-7_{CC} cells (at any selection dose) showed no resistance to taxanes or to TNFα. MCF-7_{TXT} cells at selection dose 10 produced >200-fold more TNFα than MCF-7_{CC} cells at that passage number (FIG. 4). As shown in FIG. 7, sTNFα can bind to two cell surface receptors: one with a higher affinity for TNFα (TNFR1, which possesses a death effector domain (DED) and promotes cellular apoptosis), and one with a lower affinity (TNFR2, which lacks a DED and promotes expression of NF-κB-dependent survival genes) [45]. While MCF-7_{TXT} cells above dose level 9 have high TNFR2 levels, they have dramatically reduced levels of TNFR1 (FIG. 5) suggesting that docetaxel resistance was accompanied by a shift from TNF-dependent apoptosis to TNF- and NF-κB-dependent survival. Indeed, MCF-7_{TXT} cells above dose level 9 show strong activation of NF-κB. Supporting the above findings, it was observed that both a TNFR2 neutralizing antibody and the NF-κB inhibitor SN-50 induced a 3- and 8-fold increase in docetaxel cytotoxicity in MCF-7_{TXT} cells, respectively. Collaborative microarray-based studies with functional interaction (FI) network-based analysis [46] revealed consistent changes in the expression of networks of TNF-αnd NF-κB-related genes upon acquisition of taxane resistance in breast (MCF-7, MDA-MB-231) and ovarian (A2780) tumour cells (FIG. 6). The vast majority of such genes promote TNF-mediated NF-κB activation (Table 2).

TABLE 2
TNFα- and NF-κB-related genes associated with the acquisition of docetaxel resistance in MCF-7 breast
tumor cells, MDA-MB-231 breast tumor cells, and A2780 ovarian carcinoma cells. Full genome oligo-
based microarray experiments were performed comparing differences in gene expression between wildtype
and docetaxel resistant MCF-7 cell lines. Differentially expressed genes were then classified into various
functional interaction networks. Information on the identities and roles of genes associated with TNFα
signaling are presented in tabular form, with particular emphasis on the products of genes that are known
to play a role in the induction of NF-κB-dependent survival genes or in the inhibition of apoptosis.

| | | | |
|---|---|---|---|
| | Direction and | | |
| | Magnitude | | |
| | of Change in | | |
| Gene | Expression* | Role of Gene Product | Refs |

Expression changes for TNFα- or NF-κB-related genes in MCF-7_{TXT10}
cells relative to MCF-7_{CC10} cells

| | | | |
|---|---|---|---|
| TNF | increased | Binds to TNFR1 to promote cell death and to TNFR2 to | [69;70] |
| | 12.6, 1.43 | stimulate expression of NF-κB-dependent survival genes | |
| TNFSF10 | increased | TNF-like ligand that binds to DcR2 to activate NF-κB- | [71] |
| | 6.88 | dependent survival genes; also called TRAIL | |
| TNFRSF19 | increased | Member of the TNF receptor family that binds to lymphotoxin | [72] |
| | 16.5, 1.64 | alpha activates NF-κB-mediated transcription | |
| TNFRSF14 | increased, | TNF receptor family member, interacts with members of the TNFR- | [73] |
| | 4.24 | associated factor (TRAF) family and activates NF-κB/AP-1 | |
| TNFSF13 | increased, | Binds to TNFRSF13B to promote NF-κB activation; | [74] |
| | 3.07 | aka APRIL | |
| BIRC3 | increased, | Member of a family of proteins that inhibits apoptosis by | [75] |
| | 52.2 | binding to TNFR-associated factors TRAF1 and TRAF2 | |
| TLR2 | increased, | Stimulates NF-κB activation | [76] |
| | 5.19 | | |
| TRIM38 | increased, | Removes Lys63-linked ubiquitin chains from TRAF2 | [77] |
| | 4.22, 7.28 | and TRAF6, negatively regulating NF-κB activity | |
| TNFRSF6B | decreased, | Member of the TNF receptor super family, which upon binding | [78] |
| | −2.02 | of TNF inhibits cell proliferation and induces apoptosis | |
| TRAF3IP2 | decreased, | Associates with and activates I-κB kinase, leading to | [79] |
| | −2.14 | the liberation of NF-κB from its complex with I-κB | |

Expression changes for TNFα- or NF-κB-related genes in A2780_{DXL}
cells relative to A2780 cells, MTD

| | | | |
|---|---|---|---|
| TNFSF13 | increased, | Binds to TNFRSF13B to promote NF-κB activation; | [74] |
| | 2.10 | aka APRIL | |
| TNFRSF11B | increased, | Decoy receptor for the TNF-related apoptosis-inducing ligand | [80] |
| | 2.01 | TRAIL; Confers resistance to TNF-and TRAIL-induced apoptosis | |
| CARD14 | increased, | Associates with guanylate kinase members that | [81] |
| | 2.19, 1.31 | interact with BCL10 and activate NF-κB | |
| IRAK4 | increased, | Required for the optimal transduction of IL-1-induced signals, | [82] |
| | 3.70, 3.86 | including the activation of IRAK-1, NF-κB, and JNK | |
| TLR1 | increased, | Acts via MYD88 and TRAF6 to stimulate NF-κB activation, | [83] |
| | 2.79 | cytokine secretion, and the inflammatory response | |
| TLR6 | increased, | Also acts via MYD88 and TRAF6 to stimulate NF-κB | [83] |
| | 2.26, 1.57 | activation, cytokine secretion, and the inflammatory response | |
| BIRC3 | increased, | Member of a family of proteins that inhibits apoptosis by | [75] |
| | 8.83 | binding to TNFR-associated factors TRAF1 and TRAF2 | |
| RFFL | increased, | Endosome-associated ubiquitin ligase for RIP and | [84] |
| | 2.15, 2.13 | regulates TNF-induced NF-κB activation | |
| TRAF3IP2 | increased, | Associates with and activates I-κB kinase, leading to | [79] |
| | 2.16 | the liberation of NF-κB from its complex with I-κB | |
| SMPD3 | decreased, | Translocates to the plasma membrane in response to | [85] |
| | −1.59, − [symbol] | TNFα in a time- and dose-dependent manner | |
| SOX9 | decreased, | Protein whose expression and activity is negatively | [86] |
| | −30.8 | regulated by TNF-dependent NF-κB activation | |

Expression changes in TNFα- or NF-κB-related genes in docetaxel-resistant
MB-231 cells relative to wildtype MB-231 cells

| | | | |
|---|---|---|---|
| CAST | increased, | Inhibits degradation of NF-κB to prolong NF-κB | [87] |
| | 2.44 | activation | |
| BCL10 | increased, | Potent activator of NF-κB activity | [88] |
| | 3.48 | | |
| RPS6KA3 | increased, | Phosphorylates I-κB and activates NF-κB in response | [89] |
| | 5.95 | to TNF | |
| MAP2K5 | increased, | A survival protein highly expressed in MCF-7 breast | [90] |
| | 3.54 | tumor cells resistant to etoposide and TNFα | |
| MKNK2 | increased, | Promotes TNFα biosynthesis at the post- | [91] |
| | 1.76 | transcriptional levels | |
| TNFRSF10B | decreased, | Also known as TRAIL receptor 2; stimulates apoptosis | [92] |
| | −1.67 | via FADD | |
| FADD | decreased, | A component of the caspase 8-activating complex | [93] |
| | −2.00 | induced by TNFα binding to TNFR1 | |
| CRADD | decreased, | An adaptor protein that promotes TNFα-induced apoptosis | [94] |
| | −2.43 | through interaction with the TNFR1-interacting protein RIP | |
| TRAF1 | decreased, | Negatively regulates TNFR2's ability to promote cell | [95] |
| | −2.39 | proliferation and NF-κB activation in T cells | |
| MKNK1 | decreased, | Promotes TNFα-mediated mRNA degradation | [96] |
| | −2.00 | | |

*Some columns have 2 numbers because the microarray had two probes for the same gene. Therelative change in expression (increased or decreased) is provided for both probes on the microarray. [symbol]
indicates data missing or illegible when filed

The mechanism for resistance to taxanes and TNFα in MCF-7_{TAX-1} cells may be different in that TNFR1 levels are unchanged and paclitaxel-induced TNFα production is repressed. The circumvention of TNFα's ability to stimulate TNFR1-induced cytotoxicity in MCF-7_{TAX-1} cells must be due to defects in receptor function (rather than expression) or defects downstream of the receptor. MCF-7_{TAX-1} cells are also high expressors of Abcb1 [17], which also contributes to taxane resistance by reducing paclitaxel accumulation (and hence TNFα induction) in cells. The above findings provide strong evidence that taxanes induce NF-κB-mediated TNFα production and suggests that resistance to taxanes may involve a repression of drug-induced TNFα production, a suppression of TNFα's ability to promote programmed cell death, and/or the activation of TNFα-dependent survival pathways (see model in FIG. 7).

Docetaxel Increases sTNFα Production in MCF-7_{CC} and A2780 Cells:

MCF-7_{CC} and A2780_{CC} cells secreted low levels of sTNFα (1.69×10⁻¹⁸±0.40×10⁻¹⁸ g/cell and 3.02×10⁻¹⁸±0.28×10⁻¹⁸ g/cell, respectively). These levels were not significantly changed when cells were treated with 0.1 to 1 nM docetaxel. In contrast, media extracted from MCF-7_{CC} cells treated with 3 nM docetaxel produced significantly elevated levels of sTNFα (FIG. 1A). A2780 cells produced even greater amounts of TNFα in response to docetaxel (FIG. 1A). Interestingly, the taxane paclitaxel (at concentrations 15 nM) induced even higher levels of sTNFα production than docetaxel in A2780 cells (FIG. 1B). Given the stronger induction of TNFα by docetaxel in A2780 cells, it was then assessed whether upstream mechanisms responsible for TNFα induction in A2780 cells were similar to that of macrophages. Comparable to the induction of TNFα expression by lipopolysaccharides in macrophages [104], it was observed that TNFα induction by docetaxel in A2780 cells was NF-κB-dependent, since an inhibitor of this transcription factor (SN-50) significantly reduced the induction of TNFα by docetaxel (FIG. 1C). The basal amount of sTNFα production and the magnitude of docetaxel-induced sTNFα production varied between experiments (compare FIGS. 1A and 1C for 45 nM docetaxel). Nevertheless, the sTNFα levels were consistently and substantially higher in cells treated with taxanes. The extent of TNFα induction by the taxanes appeared to decrease at higher docetaxel concentrations, possibly due to other deleterious effects of these agents on cells at the higher doses.

### Selection of MCF-7 Cells in Increasing Concentrations of Docetaxel Results in Acquisition of Progressive Docetaxel Resistance Above a Threshold Dose:

Increasing exposure of MCF-7 cells to docetaxel up to a concentration of 1.1 nM (dose 8, MCF-7_{TXT8} cells) did not affect docetaxel sensitivity (FIG. 2). However, selection to 3.33 nM docetaxel (dose 9, MCF-7_{TXT9} cells) resulted in an 11.4-fold resistance to docetaxel. Above this threshold, resistance factors increased to 16.6, 32.8, and 184 for cells selected to final docetaxel concentrations of 5 nM (dose 10, MCF-7_{TXT10} cells), 15 nM (dose 11, MCF-7_{TXT11} cells) and 45 nM (dose 12, MCF-7_{TXT12} cells), respectively. Interestingly, MCF-7_{TXT} cells exhibited an even greater cross-resistance to paclitaxel, with resistance factor of 148 and 251 at selection doses 11 and 12, respectively [23]. The resistance factor for MCF-7 cells selected for resistance to paclitaxel at the maximally tolerated dose (MCF-7_{TAX-1} cells) was 42. These cells also exhibited strong cross resistance to docteaxel (46-fold) [17]. In contrast, ovarian A2780 cells could be selected for resistance to considerably higher concentrations of docetaxel. A2780_{DXL} cells at their maximally tolerated dose (405 nM) exhibited ˜4000-fold resistance to docetaxel.

Effects of Docetaxel on sTNFα in MCF-7_{CC} and MCF-7_{TXT} Cell Lines:

MCF-7_{CC} and MCF-7_{TXT8} cells secreted low amounts of TNFα (11.5×10⁻¹⁸±0.4×10⁻¹⁸ g/cell and 5.5×10⁻¹⁸±1.4×10⁻¹⁸ g/cell, respectively). When these cell lines were exposed to 50 nM docetaxel, no significant difference in sTNFα secretion was observed (FIG. 4A). In contrast, untreated MCF-7_{TXT9} and MCF-7_{TXT10} cells secreted 31.8-fold and 18.2-fold higher levels of sTNFα than MCF-7_{CC} cells (p<0.0001), and addition of 50 nM docetaxel increased sTNFα production a further 1.62-fold and 1.27-fold, respectively (p<0.01). sTNFα levels returned to basal levels in MCF-7_{TXT11} and MCF-7_{TXT12} cells, even following treatment with 50 nM docetaxel (basal levels being levels in MCF-7_{CC}). No differences in sTNFα levels were observed between MCF-7_{CC} and MCF-7_{TAX-1} cells, in the presence or absence of docetaxel. TNFα transcript levels in MCF-7_{TXT10} cells (relative to S28 expression) was 198.5±30.5 higher than the levels of this transcript in MCF-7_{CC} cells (FIG. 4B), suggesting that elevated secretion of sTNFα is likely due to dramatically increased expression of TNFα transcripts and protein.

### MCF-7_{TXT} and MCF-7_{TAX-1} Cells are Resistant to TNFα-Induced Cytotoxicity

TNFα (10 ng/ml) reduced colony formation in a clonogenic assay by 79.8±6.0% and 66.6±1.7% for MCF-7_{CC} and MCF-7_{TXT8} cells, respectively (p<0.0001) (FIG. 3A). In contrast, MCF-7_{TXT9}, MCF-7_{TXT10}, MCF-7_{TXT11}, and MCF-7_{TAX-1} cells all had similar levels of colony formation in the absence or presence of 10 ng/mL TNFα, indicating substantial TNFα resistance. TNF actually increased colony formation in MCF-7_{TXT12} cells, possibly due a high level of activation of growth and survival pathways in these cells at the highest selection dose, some of which are TNFα-dependent. The cells lines were also cultured in the presence of varying concentrations of TNFα. Colony formation was very strongly reduced in MCF-7_{CC} cells in the presence of 50 or 100 ng/mL TNFα (p<0.0001) (FIG. 3B). Reductions in colony formation were much smaller for MCF-7_{TXT10} cells treated with 50 ng/mL or 100 ng/mL TNFα, again indicating resistance to TNFα cytotoxicity in docetaxel-resistant cells. MCF-7_{TAX-1} cells treated with 10 ng/mL TNFα formed similar numbers of colonies as untreated cells, suggesting these cells were also resistant to TNFα. However, TNFα concentrations of 50 or 100 ng/ml TNFα induced strong reductions in colony formation relative to MCF-7_{TXT10} cells, suggesting greater resistance to TNFα in the former cell line than the latter.

### TNFR1 Protein Levels (but not Transcript Levels) are Reduced Upon Acquisition of Docetaxel Resistance in MCF-7 Cells:

Unlike TNFR2, the levels of TNFR1 protein (as measured in immunoblotting experiments) decreased upon acquisition of docetaxel resistance at dose 9 (MCF-7_{TXT9} cells) and remained low in MCF-7_{TXT10} and MCF-7_{TXT12} cells (FIG. 5A). Interestingly, reverse transcription quantitative PCR (RTqPCR) analysis revealed no significant differences in TNFR1 transcript expression between these cell lines (FIG. 5B). Similar soluble TNFR1 (sTNFR1) levels were observed in MCF-7_{CC} and MCF-7_{TXT8} cells (FIG. 5C), although levels decreased in MCF-7_{TXT9} and MCF-7_{TXT10} cells (p<0.001). sTNFR1 levels then returned to those of MCF-7_{TXT8} cells as docetaxel selective pressure was increased.

### Induction of Docetaxel Resistance in MCF-7 Cells Through Application of a TNFR1 Neutralizing Antibody:

Significant differences in colony formation were observed between TNFR1 neutralizing antibody-treated MCF-7_{CC} cells and untreated cells when incubated with 1.23 nM (p<0.0001), 0.41 nM (p=0.0002), 0.14 nM (p=0.0006) and 0.046 nM (p<0.0001) docetaxel (FIG. 5D). Non-linear regression curve-fitting programs revealed that MCF-7_{CC} cells incubated with the TNFR1 neutralizing antibody were about 2.25-fold more resistant to docetaxel than untreated cells, consistent with a role for the TNFα pathway in docetaxel cytotoxicity.

### Activation of NF-κB Upon Acquisition of Docetaxel Resistance

Unlike MCF-7_{TAX-1} cells, MCF-7_{TXT10} cells had 35% lower IκB levels than MCF-7_{CC} cells (p=0.03) (FIG. 9A). Measurement of NF-κB binding in nuclear extracts from MCF-7 and MCF-7_{TXT8} cells revealed low binding of NF-κB p65 and p50 subunits to the NF-κB transcription factor binding site (FIGS. 9B and 9C). In contrast, nuclear extracts from MCF-7_{TXT9} and MCF-7_{TXT10} cells exhibited >3-fold higher levels of subunit binding to the NF-κB sequence compared to equivalent extracts from MCF-7_{CC} cells (p<0.05). This binding was reduced as cells were exposed to higher docetaxel selection doses. Interestingly, 50 nM docetaxel induced even higher levels of p65 and p50 subunit binding in MCF-7_{CC} and MCF-7_{TXT} cells, except when docetaxel selection doses were >15 nM (dose 11 and 12).

Promotion of TNFα Cytotoxicity in MCF-7_{TXT10} cells by cycloheximide or a TNFR2 Neutralizing Antibody

As previously observed, exposure of MCF-7_{CC} cells to 10 ng/mL TNFα strongly decreased colony formation in a clonogenic assay while MCF-7_{TXT10} cells exhibited significant resistance to TNFα (FIG. 10A). The addition of the protein synthesis inhibitor cycloheximide 5 μg/mL restored TNFα's ability to be cytotoxic to MCF-7_{TXT10} cells, while having only a small additional effect on TNFα cytotoxicity in MCF-7_{CC} cells. These observations suggested that a protein, possibly NF-κB, is critical for maintaining resistance to TNFα.

To test the above hypothesis, and since NF-κB is activated upon TNFα binding to TNFR2, resulting in enhanced expression of survival genes [97], it was theorized that docetaxel cytotoxicity might be increased in MCF-7_{TXT10} cells upon addition of a TNFR2 neutralizing antibody or an inhibitor of NF-κB function. Supporting this conjecture, a greater reduction in colony formation for TNFR2 neutralizing antibody-treated cells than untreated cells when treated with 41.2 nM (p=0.0007), 13.7 nM (p=0.005), 4.5 nM (p=0.006) or 1.7 nM (p=0.01) docetaxel was observed (FIG. 10B). Non-linear regression curve-fitting for 3 independent experiments revealed that the TNFR2 neutralizing antibody rendered MCF-7_{TXT10} cells 2.13-fold more sensitive to docetaxel than untreated cells. Moreover, as shown in FIG. 10C, the peptide SN-50, which contains the nuclear localization signal of NF-κB and thus blocks the transcription factor's translocation to the nucleus [60], increased docetaxel cytotoxicity to an even greater degree in MCF-7_{TXT10} cells (7.1-fold). In contrast, a control peptide (SN-50M), in which critical basic amino acids within the nuclear localization signal are replaced with uncharged amino acids, had no effect on docetaxel sensitivity (FIG. 10C).

Network-Based Analysis of Genes Associated with the Acquisition of Docetaxel Resistance:

Assessment of microarray data using an FI network approach revealed 2235 genes that were differently expressed between parental and docetaxel-resistant MCF-7 breast cancer cell lines (fold-change >2.0 and FDR ≦0.05). Of these, 834 (37.3%) were in the FI network and hierarchical clustering reduced this to a set of 753 of the most interconnected candidates. This gene set was then used for further analyses. The average shortest distance calculation showed that genes in this set were linked together much more tightly than would be expected by chance alone (p<0.001), indicating that these differentially expressed genes occupy a small corner of the large FI network space. A sub-network was built from these 753 genes by adding the minimum number of linker genes required to form fully connected networks involving these genes. The resulting networks consisted of 938 genes, 185 of which were linkers. A Markov clustering algorithm was then used to identify clusters of proteins (coded by the genes) that are highly interconnected with each other and less connected to the outside world. This algorithm identified 14 clusters consisting of >20 genes, including a cluster of 22 TNF-associated genes and 8 linkers (FIG. 6A).

An identical approach was used to identify clusters of differentially expressed genes between wildtype and docetaxel-resistant A2780 ovarian carcinoma cells. Out of 955 genes that were differentially expressed between the two cell lines, a network of 11 TNF-related genes and 3 linkers was identified (FIG. 6B). When the same approach was used to identify networks of genes differentially expressed between docetaxel-sensitive and docetaxel-resistant MDA-MB-231 cells (data obtained from GEO, accession numbe GSE28784), a cluster of 22 TNF-related genes and 3 linkers was identified (see FIG. 6C).

### Confirmation of Changes in the Expression of TNFα-Dependent Genes by RTqPCR:

The expression of a select number of genes within the above-identified TNFα signaling networks was quantitatively assessed by RTqPCR. As shown in FIG. 11, there was generally a strong concordance between changes in gene expression identified by microarray analysis and those determined by RTqPCR (12 of 14 gene expression changes assessed). Six TNFα-dependent genes were confirmed to have altered expression upon selection of MCF-7 cells for resistance to docetaxel, including TNFSF13, TNFSF10, TLR6, TNFAIP3, TNFSF14, and BIRC3 (the latter two genes being upregulated 30-fold, and 21-fold, respectively). Three of these genes were also upregulated in A2780_{D×L} cells (BIRC3, TLR6, and TNFSF10, which increased expression almost 300-fold).

Quantitative PCR detected a difference in TNF alpha transcript expression between MCF-7TXT10 and MCF-7CC10 cells near 200-fold.

### Relevance of the TNFα and NF-κB Pathways in Clinical Response to Taxanes in Cancer Patients:

Interestingly, docetaxel concentrations required to induce significant TNFα production in cells are well within the range of plasma levels of docetaxel observed in breast cancer patients following docetaxel infusion (10-75 nM) [47].

### Discussion

Although taxanes are known to inhibit cell division by preventing microtubule depolymerization and inducing multi-nucleation [13, 105], it is unclear whether these are their sole mechanisms of tumor cell growth arrest/death in vitro and in vivo. Paclitaxel has been shown to increase sTNFα release from murine macrophages [106, 107], although the levels used in those studies would be unachievable in patients, and docetaxel had no effect on TNFα expression in the same study. In this study, it is shown that docetaxel (at concentrations between 3 to 45 nM) can stimulate TNFα production and sTNFα release from both breast and ovarian tumor cells. Such concentrations are clearly in the range of plasma levels of docetaxel in breast cancer patients following docetaxel infusion (10-75 nM) [15] and are likely sufficiently high to induce TNF expression in even poorly vascularized tumours. This newly identified TNF-dependent mechanism of docetaxel action may also account for its reported immunomodulatory activity [108, 109]. In addition, it is shown that paclitaxel treatment (at 5 and 15 nM concentrations) can dramatically increase sTNFα release from ovarian tumor cells.

These results also illustrates that the acquisition of docetaxel resistance in breast tumor cells temporally correlates with increased production and release of sTNFα from cells, despite the ability of sTNFα to be cytotoxic to cells [110]. However, the onset of docetaxel resistance in MCF-7 cells (at docetaxel selection doses ≧3.33 nM) also correlated with strongly reduced levels of TNFR1 intracellular protein, which would block TNFα's ability to induce cell death. Although the mechanism responsible for TNFR1 reduction remains undefined, neither changes in TNFR1 transcript levels nor increased levels of sTNFR1 in the media were found, suggesting that the receptor was not shed from cells by the ADAM-17 protease [111]. In fact, MCF-7_{TXT9} and MCF-7_{TXT10} cells exhibited decreased levels of sTNFR1 in the medium in which it was grown. It is possible that increased levels of sTNFα produced by these cells bound to sTNFR1 in the medium, preventing its detection by the TNFR1 antibody. Taken together, these findings suggest that downregulation of TNFR1 occurs post-transcriptionally, either due to reduced translation of the TNFR1 transcript or increased TNFR1 proteolysis.

A recent study [112] found that TNFα or paclitaxel induced NF-κB activity in C2C12 myotubes. However, paclitaxel did not induce increased TNFα production and inhibition of TNFR1 blocked TNFα-induced NFκB activation but did not abolish paclitaxel-induced NF-κB activity [112]. It is important to note that in these studies, TNFα levels were assessed only 4 hours following treatment with paclitaxel (10 nM to 10 μM).

While docetaxel selection doses between 3 and 5 nM resulted in highly elevated sTNFα production, higher selection doses (≧15 nM) did not. This was despite the drug's ability to induce TNFα production in wildtype cells over a large concentration range (FIG. 1). This may be explained by the increased expression of the Abcb1 drug transporter and reduced docetaxel uptake that we observed in MCF-7_{TXT11} and MCF-7_{TXT12} cells. Expression was maximal at the highest selection doses (≧15 nM) [113]. Docetaxel may accumulate at sufficient concentrations to induce production of sTNFα in MCF-7_{TXT9} and MCF-7_{TXT10} cells. However, at or above 15 nM docetaxel, MCF-7_{TXT} cells exhibit reduced drug uptake, such that docetaxel accumulation may be insufficient to stimulate TNFα production.

The mechanism for resistance to taxanes and TNFα in MCF-7_{TAX-1} cells appears to differ from MCF-7_{TXT} cells. TNFR1 levels were equivalent in MCF-7_{TAX-1} and MCF-7_{CC} cells and IκB levels were also unchanged during selection for paclitaxel resistance (FIG. 9). Since only cells exposed to the maximally tolerated dose of paclitaxel were retained during selection of MCF-7_{TAX-1} cells, it is likely that cells selected at lower doses could have exhibited elevated production of TNFα and TNFα-mediated NF-κB activation. However, survival by circumventing TNFα's ability to stimulate TNFR1-induced cytotoxicity must lie downstream of the receptor. MCF-7_{TAX-1} cells are also high expressors of Abcb1 [17]. Interestingly, another paclitaxel-resistant MCF-7 cell line (MCF-7_{TAX-2} cells) [18] retained sensitivity to TNFα, suggesting that defects in the TNFα pathway are not critical for taxane resistance in vitro. Nevertheless, three of the four taxane-resistant cell lines exhibited alterations in TNFα signaling and docetaxel has been shown to increase sTNFα levels in both breast and ovarian tumor cells, identifying a mechanism of taxane cytotoxicity and resistance.

To provide further support for a general involvement of the TNFα pathway in docetaxel cytotoxicity and in the induction of docetaxel resistance, it was shown in this study that selection of breast and ovarian tumor cells for resistance to docetaxel results in changes in the expression of networks of genes related to TNFα signaling (FIG. 6 and Table 2). Quite strikingly, the vast majority of the upregulated genes depicted in Table 2 code for proteins that are TNF ligand family members, TNF receptor family members, TNF receptor-associated proteins, TNF-dependent activators of NF-κB, or proteins that help promote degradation of the inhibitor of NF-κB (I-κB). Other upregulated genes are TNF-dependent inhibitors of apoptosis. Downregulated genes code for proteins that inhibit the activation of NF-kB or promote apoptosis. The net effect of the changes in gene expression would thus be to promote TNF's ability to augment NF-κB-dependent cell survival, while blocking its ability to induce tumour cell death via activation of TNFR1. The findings of this study may have significant clinical relevance. A paper presented at the 26^{th} annual meeting of the European Association of Urology in 2011 [114] revealed that serum levels of proinflammatory cytokines, including TNFα, increased two days after administration of docetaxel to patients with castration-resistant prostate cancer. Interestingly, these changes in cytokine expression correlated with the induction of apoptosis and with clinical response. In addition, a study presented recently at the American Association for Cancer Research 101^{st} Annual Meeting [52] revealed that in patients with serous epithelial ovarian carcinoma, pre-treatment tumour expression of various genes within the TNFα and NF-κB signalling networks could be used to distinguish between responders and nonresponders of paclitaxel/carboplatin chemotherapy. It has also been shown in a small study involving patients with locally advanced breast cancer that pretreatment tumour levels of nuclear (activated) NF-κB could be used to distinguish between responders and non-responders to neoadjuvant anthracycline- and/or taxane-based chemotherapy regimens [48]. These studies strongly support the likely clinical significance of findings. For example, since TNFα has been shown to reduce tumor vascularization in mice through its effects on TNFR1-expressing endothelial cells [68], docetaxel's reported ability to affect tumor angiogenesis may be through an ability of the drug to promote sTNFα-mediated decreases in tumor vascularization. Moreover, one of the well-established dose-limiting toxicities associated with docetaxel chemotherapy in breast cancer patients is fatigue [116] and high TNF levels have been shown to correlate with fatigue onset in cancer patients [117]. Given the findings of docetaxel-induced TNFα production, perhaps these two phenomena are linked. Finally, a previous clinical study used a TNF-decoy receptor (entanercept) to permit patients to tolerate higher doses of docetaxel without significant toxicity [118]; however, given the disclosed findings, it is not surprising that these blockers would create a greater tolerance to docetaxel, unfortunately at the likely expense of lesser anti-tumor efficacy. These findings further question the utility of administering docetaxel to cancer patients on TNFα blockers for treatment of co-morbid inflammatory diseases.

It is unknown if other chemotherapies require TNFα to induce death. Milner et al., 2002 (Cell Death and Differentiation (2002) 9:287-300) found increased TNF alpha production in A2780 ovarian tumour cells in response to cisplatin, doxorubicin, and vinblastine. Topotecan had no effect on TNF alpha levels. However neutralizing TNF did not block apoptosis.

Zembala et al., 1993 showed no effect of chemotherapy (5FU, doxorubicin, and mitomycin C) on monocyte TNF levels in gastric cancer patients.

### Example 3

The RNA from core biopsies collected from 93 locally advanced breast cancer patients taken prior to, during, and post chemotherapy will be used to monitor the expression of TNFα-related genes by RTqPCR during treatment. Using this data and data from a recently completed Agilent™ full genome microarray study, pre-, mid-, or post-treatment expression of TNFα or related transcripts [identified in vitro (Table 2) or in the MA-22 patient microarray data will be correlated with various measures of clinical response or toxicity/resistance to epirubicin/docetaxel chemotherapy (as described below). In some patients, chemotherapy treatment strongly reduced tumour RNA quantity and quality, and low mid-treatment RNA integrity was associated with a pCR post-treatment [49]. In order to account for this reduction in RNA quantity and quality, the expression of all transcripts by RTqPCR will be normalized relative to the expression of seven reference genes (HMBS, HPRT1, MRPL19, PUM1, RPL13A, SDHA, and SF3A1). TNFR1 and TNFR2 transcript expression will also be assessed.

In addition to transcript profiling, tumour tissue microarrays from patients prior to, during, and post-treatment will be assessed by immunohistochemical staining for expression of TNFα, activated (nuclear) NF-κB, TNFR1, and TNFR2 proteins. Tumour RTqPCR and immunohistochemical microscopy data will be assessed) for correlation of tumour expression of select TNFα-related transcripts and/or proteins with: 1) patient treatment (total docetaxel and epirubicin doses received), 2) toxicity via common grade 3/4 adverse events (fatigue, neutropenia), 3) new and continuing co-morbid disease, 4) concomitant medication (such as the TNFR1 antagonist Enbrel), or various measures of clinical response, including 5) the RECIST criteria [50], 6) clinical response, 7) pathologic complete response, 8) tumour extent.

While nonresponding tumours may consistently lack the ability to produce TNFα when exposed to epirubicin/docetaxel chemotherapy (possibly due to impaired uptake of the chemotherapy drugs into such tumours), it is possible these tumours may exhibit: a) other defects in the ability of the chemotherapy regimen to activate TNFα-mediated cell death pathways and/or b) have strong activation of TNFα-related NF-κB-dependent survival pathways. Thus, the microarray and RTqPCR gene profiling data described above may provide significant insight into TNFα-related mechanisms that impact on patient response to taxane-based chemotherapy. Partek Genomics Suite™ and BRB array tools will be used to analyze and cluster the genomic data from these studies in order to relate tumour gene expression and patient characteristics data to clinical response (e.g. toxicity and/or resistance to treatment). Bioinformatic tools will be used to identify gene pathways which will be multivariately tested with inclusion/exclusion of ‘k’ genes in a pathway and chi-squared tests of their effect (X²₍ₖ₎). Where possible, continuous variables and endpoints will be used to maximize power with our small sample sizes.

In an additional approach, microarray data, curated pathway databases, and FI network analysis [46] will be used to reduce the above large list of genes differentially expressed genes between tumour subtypes, between pre- and mid-treatment time points, and between chemo-responsive and chemo-resistant tumours (as defined above) into networks of differentially expressed genes associated with response or resistance to taxane-based chemotherapy regimens.

The ability of the expression of TNFα-related network members to predict chemotherapy response (including for example at specific treatment times) in MA.22 patients will be confirmed using high throughput RTqPCR (controlling for chemotherapy-dependent RNA degradation in some patients as described above). A Bayes Naive classifier will be built to distinguish between chemotherapy-responsive and nonresponsive tumours. Several features (alone or in combination) that may affect the efficacy of the classifier will be assessed: (1) TNFα transcript or protein levels; and (2) expression values of each gene in the TNFα/NF-κB-related clusters identified in vitro (see FIG. 6 and Table 2) and data from a microarray study on MA.22 patients. The pre-treatment gene profiling of MA.22 tumours by microarray analysis will also enable the use of non-supervised hierarchical clustering algorithms to classify each tumour into specific subtypes [51]. This will, in turn, enable the assessment of whether specific subtypes of breast tumours (for example, the more chemoresponsive basal and HER2 subtypes) produce more TNFα in response to taxane treatment. In the MA.22 trial, a large percentage of patients were of the luminal and basal subtypes, with few HER2 tumours due to trial eligibility criteria.

### Example 4

Another study is being conducted, whereby fine needle aspirates (FNAs) are being obtained from 10 patients with locally advanced breast cancer at various time points: i) pre-treatment and at iii) 1 and iv) 2 days after administration of the first cycle of taxane chemotherapy. FNAs will also be obtained from patients v) immediately following the last cycle of taxane chemotherapy and vi) from recurrent tumours after taxane chemotherapy. All FNAs will be immediately divided into 2×100 ul aliquots and flash frozen on dry ice. RNA will be isolated from one aliquot of each FNA using Qiagen™ total RNA miRNAeasy kits. A whole cell protein extract will also be prepared from the other aliquot of each FNA by adding 11 ul of 10× Accustain™ protein extraction buffer (Sigma Chemical Company). The RNA and protein preparations at the various treatment times will be monitored for TNFα transcript (RTqPCR) and protein (ELISA) expression. Should the FNAs yield sufficient TNFα transcript or protein levels for reliable detection, FNAs will be obtained from an additional 30 patients at the above time points and also assessed for TNFα transcript and protein levels. FI network analysis (after microarray-based gene expression profiling of the above RNA preparations at the various treatment times) will then conducted and will provide an accurate assessment of whether: a) networks of TNFα- or NF-κB-related genes change expression in response to taxane chemotherapy, and b) whether the expression of one or more TNFα-related genes at various time points can be used to distinguish between patients that respond well to the regimen (pCR) and those that do not. Response will be defined as progressive disease, stable disease, partial response, complete clinical response, or pCR. Taxane response will be assessed by monitoring change in tumour size and the extent of tumour cellularity in the breast and axilla at the various treatment times.

### Example 5

IN VITRO EXPERIMENTS: Whether other chemotherapy agents used to treat breast and ovarian cancer (alone or in combination with taxanes) promote TNFα production in cell lines and in mouse xenograft experiments will be addressed. This will determine whether some chemotherapy agents may interfere with TNFα induction by taxanes. Recent in vitro data suggests that doxorubicin is another TNFα-inducing chemotherapy agent (FIG. 1) and the MA.22 data suggests that the presence of epirubicin does not block TNFα induction. Using standard ELISA methods, it will be determined whether a variety of drugs used in the treatment of breast or ovarian cancer induce TNFα production (after an incubation period of 48 h). These include cyclophosphamide, epirubicin, doxorubicin, 5-F1-uracil, and carboplatin (at concentrations from 0 to an order of magnitude above their 1050 values (concentration required to reduce colony formation by 50% in a clonogenic assay). Cellular levels of TNFα will be determined using the same ELISA kit and detergent-soluble extracts of cells using a previously published method [59]. Given that TNFR1 and TNFR2 levels determine what signaling pathways are activated by TNFα, we will also assess the levels of TNFR1 and TNFR2 in these extracts using standard immunblotting procedures.

Whether selection for resistance to the above agents compromises the TNFα induction capacity and cytotoxicity of docetaxel or the above agents will be assessed. Several cell lines are available to address this, including MCF-7 breast tumour cells selected for resistance to doxorubicin (MCF-7DOX and MCF-7DOX-2) epirubicin (MCF-7EPI), and 5-FI-uracil (MCF-75FU). A2780 ovarian tumour cells selected for resistance to carboplatin (A2780CBN), docetaxel (A2780DXL), or both carboplatin and docetaxel (A2780CBNDXL) are also available.

The TNFR1- and NF-κB-dependence of TNFα induction and cytotoxicity by taxanes and/or other drugs will be determined by conducting the above TNFα production (ELISA) and drug cytotoxicity (clonogenic) assays in the absence (PBS) or presence of a TNFR1-neutralizing antibody from R&D Systems (at 0-30 mg/ml), the TNFR1 antagonist entanercept (Enbrel®, Amgen, Inc., 0 to 30 mg/ml), a peptide (SN-50; Calbiochem Laboratories) that blocks NF-κB function by inhibiting translocation of the NF-κB complex into the nucleus [60], and a small molecule inhibitor of NF-κB [sulfasalazine (Sigma), at concentrations from 1 uM to 2 mM][61]. Whether various concentrations of recombinant TNFα, a selective TNFR1 agonist (MT1-2; 0 to 5 mg/ml; Cell Sciences, Caton, Mass.) or a selective TNFR2 antagonist (TNFR2 neutralizing antibody; R&D Systems) can increase taxane cytotoxicity in the above taxane-resistant cell lines will also be assessed.

MOUSE XENOGRAFT EXPERIMENTS: The above will also be examined in mice bearing human xenografts of breast and ovarian tumour cells. Five million MCF-7 breast and A2780 ovarian tumour cells [suspended in 0.3 ml Matrigel-MEM (Becton Dickinson) containing growth factors] will be xenografted into nu/nu mice (20-25 gm) from Charles River Laboratories. The typical tumour take rate has been between 80 and 90%. The mice will be weighed daily and tumour volumes determined every 5 days by caliper measurement using the formula for a semiellipsoid (4/3πr3/2). Upon reaching a tumour volume of 200 mm3 (˜4 weeks), the tumour bearing mice will be randomly assigned to one of the following groups: a) sacrificed immediately (Day 1 control), b) saline treatment (sham) c) paclitaxel treatment, d) paclitaxel treatment plus an additional agent. Paclitaxel (10 mg/kg i.p.) will be administered (with or without additional agents, see below) on day 1 followed by subsequent 10 mg/kg doses (i.p.) on days 14, 28, 42, and 56. These doses have been effective in reducing the growth of mouse tumour xenografts in our hands (and others [62]) and are well below the maximal tolerable dose. Where possible, animal tumours will be serially biopsied using a Bard Magnum biopsy gun fitted with a 16-gauge needle, in order to assess intra-animal and inter-animal data variability. One biopsy will be taken just prior to taxane administration and two days after drug administration for assessment of tumour TNFα transcript levels [by RTqPCR, as described previously for MA.22 patients]. Other mice will be sacrificed at the time of drug administration, and 2, 14, 28, 42, and 56 days post taxane administration [also for assessment of TNFα transcript levels by RTqPCR]. Twenty animals will be used for each treatment group listed above. An Animal Use Protocol (AUP) for the investigation of anthracyclines and taxanes in mouse xenografts of human tumour cells has been reviewed and approved by the Animal Care Committee of Laurentian University.

Taxane-resistant tumours will be established by xenografting MCF-7TXT, MCF-7TAX-1 and A2780DXL cells into immunodeficient nu/nu mice. Resistance will be confirmed in separate mouse experiments. If resistance in vivo is not achieved with these cell lines, alternatively establish taxane-resistant tumours by continuous exposure of tumour xenografts to taxanes using previously published approaches will be established[63;64]. It will then be assessed whether taxanes are unable to induce tumour TNFα production in mice bearing resistant tumours and whether such tumours exhibited elevated TNFα production. Finally, it will be assessed whether administration of 5 mg of recombinant human TNFα (R&D Systems) in 100 ul (i.p) or an equimolar concentration of the TNFR1 agonist MT1-2 or the TNFR2 neutralizing antibody (R&D Systems) can augment docetaxel's ability to reduce tumour size in wildtype and taxane-resistant tumours that retain TNFR1 expression. The above dose of TNFα has been shown to induce tumour regression in mouse xenografts [68]. Example 7

Whether taxanes (with or without) additional chemotherapy agents can promote tumour TNFα transcript expression in patients with locally advanced breast cancer and whether the pre-, mid-, or post-treatment expression of TNFα- or related genes [based on both in vitro and vivo studies] can be effectively used to predict or monitor clinical response to taxane-based chemotherapy regimens in patients with breast or ovarian cancer will be assessed. I In addition, it will be assessed whether the induced expression of TNFα and related genes by taxane-based regimens occurs preferentially in specific subtypes of breast cancer and whether these TNFα-related biomarkers are better at predicting or assessing response in these subtypes.

ii) IN VITRO AND MOUSE XENOGRAFT EXPERIMENTS: It will be addressed in cell lines and in mouse xenograft experiments whether other chemotherapy agents used to treat breast and ovarian cancer can promote TNFα production and whether selection for resistance to these agents also compromises taxane cytotoxicity and taxane-induced TNFα production. Finally, it will be assessed whether TNFR1 pathway agonists or TNFR2 pathway antagonists may help restore TNF production and/or taxane sensitivity in taxane-resistant cells and tumours that retain TNFR1 and TNFR2 expression.

### CITATIONS FOR REFERENCES REFERRED TO IN THE SPECIFICATION

[1] Canadian Cancer Society and Statistics Canada Canadian Cancer Statistcs 2012, 2012.
[2] I. Smith and S. Chua, Medical treatment of early breast cancer. I: adjuvant treatment, BMJ 332 (2006) 34-37.
[3] A. Eltahir, S. D. Heys, A. W. Hutcheon, T. K. Sarkar, I. Smith, L. G. Walker, A. K. Ah-See, and O. Eremin, Treatment of large and locally advanced breast cancers using neoadjuvant chemotherapy, Am. J. Surg. 175 (1998) 127-132.
[4] I. Smith and S. Chua, Medical treatment of early breast cancer. IV: neoadjuvant treatment, BMJ 332 (2006) 223-224.
[5] S. M. Geurts, A. M. van Altena, V. F. de, V. C. Tjan-Heijnen, L. F. Massuger, J. A. van Dijck, and A. L. Verbeek, No supportive evidence for clinical benefit of routine follow-up in ovarian cancer: a Dutch multicenter study, Int. J. Gynecol. Cancer 21 (2011) 647-653.
[6] S. Tingulstad, F. E. Skjeldestad, T. B. Halvorsen, and B. Hagen, Survival and prognostic factors in patients with ovarian cancer, Obstet. Gynecol. 101 (2003) 885-891.
[7] J. H. Jacob, D. M. Gershenson, M. Morris, L. J. Copeland, T. W. Burke, and J. T. Wharton, Neoadjuvant chemotherapy and interval debulking for advanced epithelial ovarian cancer, Gynecol. Oncol. 42 (1991) 146-150.
[8] J. Crown, M. O'Leary, and W. S. Ooi, Docetaxel and paclitaxel in the treatment of breast cancer: a review of clinical experience, Oncologist. 9 Suppl 2 (2004) 24-32.
[9] J. G. Zijlstra, J. S. de, E. G. de Vries, and N. H. Mulder, Topoisomerases, new targets in cancer chemotherapy, Med Oncol Tumor Pharmacother. 7 (1990) 11-18.
[10] D. S. Richardson and S. A. Johnson, Anthracyclines in haematology: preclinical studies, toxicity and delivery systems, Blood Rev. 11 (1997) 201-223.
[11] G. Capranico, I. P. De, S. Penco, S. Tinelli, and F. Zunino, Role of DNA breakage in cytotoxicity of doxorubicin, 9-deoxydoxorubicin, and 4-demethyl-6-deoxydoxorubicin in murine leukemia P388 cells, Cancer Res. 49 (1989) 2022-2027.
[12]. Ringel and S. B. Horwitz, Studies with RP 56976 (taxotere): a semisynthetic analogue of taxol, J Natl Cancer Inst 83 (1991) 288-291.
[13] M. Chazard, B. Pellae-Cosset, F. Garet, J. A. Soares, B. Lucidi, Y. Lavail, and L. Lenaz, Taxol (paclitaxel), first molecule of a new class of cytotoxic agents: taxanes, Bull Cancer 81 (1994) 173-181.
[14] A. U. Buzdar, S. E. Singletary, R. L. Theriault, D. J. Booser, V. Valero, N. Ibrahim, T. L. Smith, L. Asmar, D. Frye, N. Manuel, S. W. Kau, M. McNeese, E. Strom, K. Hunt, F. Ames, and G. N. Hortobagyi, Prospective evaluation of paclitaxel versus combination chemotherapy with fluorouracil, doxorubicin, and cyclophosphamide as neoadjuvant therapy in patients with operable breast cancer, J. Clin. Oncol. 17 (1999) 3412-3417.
[15] A. du Bois, H. J. Luck, W. Meier, H. P. Adams, V. Mobus, S. Costa, T. Bauknecht, B. Richter, M. Warm, W. Schroder, S. Olbricht, U. Nitz, C. Jackisch, G. Emons, U. Wagner, W. Kuhn, and J. Pfisterer, A randomized clinical trial of cisplatin/paclitaxel versus carboplatin/paclitaxel as first-line treatment of ovarian cancer, J. Natl. Cancer Inst. 95 (2003) 1320-1329.
[16] C. H. Choi, ABC transporters as multidrug resistance mechanisms and the development of chemosensitizers for their reversal, Cancer Cell Int 5 (2005) 30.
[17] B. Guo, D. J. Villeneuve, S. L. Hembruff, A. F. Kirwan, D. E. Blais, M. Bonin, and A. M. Parissenti, Cross-resistance studies of isogenic drug-resistant breast tumor cell lines support recent clinical evidence suggesting that sensitivity to paclitaxel may be strongly compromised by prior doxorubicin exposure, Breast Cancer Res Treat 85 (2004) 31-51.
[18] S. L. Hembruff, M. L. Laberge, D. J. Villeneuve, B. Guo, Z. Veitch, M. Cecchetto, and A. M. Parissenti, Role of drug transporters and drug accumulation in the temporal acquisition of drug resistance, BMC Cancer 8 (2008) 318.
[19] A. J. Primeau, A. Rendon, D. Hedley, L. Lilge, and I. F. Tannock, The distribution of the anticancer drug Doxorubicin in relation to blood vessels in solid tumors, Clin. Cancer Res. 11 (2005) 8782-8788.
[20]. F. Tannock, C. M. Lee, J. K. Tunggal, D. S. Cowan, and M. J. Egorin, Limited penetration of anticancer drugs through tumor tissue: a potential cause of resistance of solid tumors to chemotherapy, Clin. Cancer Res. 8 (2002) 878-884.
[21] H. J. Arts, D. Katsaros, E. G. de Vries, M. Massobrio, F. Genta, S. Danese, R. Arisio, R. J. Scheper, M. Kool, G. L. Scheffer, P. H. Willemse, A. G. van der Zee, and A. J. Suurmeijer, Drug resistance-associated markers P-glycoprotein, multidrug resistance-associated protein 1, multidrug resistance-associated protein 2, and lung resistance protein as prognostic factors in ovarian carcinoma, Clin. Cancer Res. 5 (1999) 2798-2805.
[22] L. Pusztai, P. Wagner, N. Ibrahim, E. Rivera, R. Theriault, D. Booser, F. W. Symmans, F. Wong, G. Blumenschein, D. R. Fleming, R. Rouzier, G. Boniface, and G. N. Hortobagyi, Phase II study of tariquidar, a selective P-glycoprotein inhibitor, in patients with chemotherapy-resistant, advanced breast carcinoma, Cancer 104 (2005) 682-691.
[23]. F. Tannock, Tumor physiology and drug resistance, Cancer Metastasis Rev. 20 (2001) 123-132.
[24] A. M. Parissenti, S. L. Hembruff, D. J. Villeneuve, Z. Veitch, B. Guo, and J. Eng, Gene expression profiles as biomarkers for the prediction of chemotherapy drug response in human tumour cells, Anticancer Drugs 18 (2007) 499-523.
[25] V. Guarneri, K. Broglio, S. W. Kau, M. Cristofanilli, A. U. Buzdar, V. Valero, T. Buchholz, F. Meric, L. Middleton, G. N. Hortobagyi, and A. M. Gonzalez-Angulo, Prognostic value of pathologic complete response after primary chemotherapy in relation to hormone receptor status and other factors, J. Clin. Oncol. 24 (2006) 1037-1044.
[26] K. S. Albain, W. E. Barlow, P. M. Ravdin, W. B. Farrar, G. V. Burton, S. J. Ketchel, C. D. Cobau, E. G. Levine, J. N. Ingle, K. I. Pritchard, A. S. Lichter, D. J. Schneider, M. D. Abeloff, I. C. Henderson, H. B. Muss, S. J. Green, D. Lew, R. B. Livingston, S. Martino, and C. K. Osborne, Adjuvant chemotherapy and timing of tamoxifen in postmenopausal patients with endocrine-responsive, node-positive breast cancer: a phase 3, open-label, randomised controlled trial, Lancet 374 (2009) 2055-2063.
[27] R. C. Young, Early-stage ovarian cancer: to treat or not to treat, J. Natl. Cancer Inst. 95 (2003) 94-95.
[28] A. Hurria, M. T. Fleming, S. D. Baker, W. K. Kelly, K. Cutchall, K. Panageas, J. Caravelli, H. Yeung, M. G. Kris, J. Gomez, V. A. Miller, G. D'Andrea, H. I. Scher, L. Norton, and C. Hudis, Pharmacokinetics and toxicity of weekly docetaxel in older patients, Clin. Cancer Res. 12 (2006) 6100-6105.
[29] J. A. Sparano, M. Wang, S. Martino, V. Jones, E. A. Perez, T. Saphner, A. C. Wolff, G. W. Sledge, Jr., W. C. Wood, and N. E. Davidson, Weekly paclitaxel in the adjuvant treatment of breast cancer, N. Engl. J. Med. 358 (2008) 1663-1671.
[30] N. I. Marupudi, J. E. Han, K. W. Li, V. M. Renard, B. M. Tyler, and H. Brem, Paclitaxel: a review of adverse toxicities and novel delivery strategies, Expert. Opin. Drug Saf 6 (2007) 609-621.
[31] T. Sorlie, C. M. Perou, R. Tibshirani, T. Aas, S. Geisler, H. Johnsen, T. Hastie, M. B. Eisen, R. M. van de, S. S. Jeffrey, T. Thorsen, H. Quist, J. C. Matese, P. O. Brown, D. Botstein, P. E. Lonning, and A. L. Borresen-Dale, Gene expression patterns of breast carcinomas distinguish tumor subclasses with clinical implications, Proc. Natl. Acad. Sci. U. S. A 98 (2001) 10869-10874.
[32] L. A. Carey, E. C. Dees, L. Sawyer, L. Gatti, D. T. Moore, F. Collichio, D. W. Ollila, C. I. Sartor, M. L. Graham, and C. M. Perou, The triple negative paradox: primary tumor chemosensitivity of breast cancer subtypes, Clin. Cancer Res. 13 (2007) 2329-2334.
[33] D. Tripathy, Gemcitabine in breast cancer: future directions, Clin. Breast Cancer 3 Suppl 1 (2002) 45-48.
[34] W. A. Weber, M. Schwaiger, and N. Avril, Quantitative assessment of tumor metabolism using FDG-PET imaging, Nucl. Med. Biol. 27 (2000) 683-687.
[35] S. J. Kim, S. K. Kim, E. S. Lee, J. Ro, and S. Kang, Predictive value of [18F]FDG PET for pathological response of breast cancer to neo-adjuvant chemotherapy, Ann. Oncol. 15 (2004) 1352-1357.
[36] G. M. McDermott, A. Welch, R. T. Staff, F. J. Gilbert, L. Schweiger, S. I. Semple, T. A. Smith, A. W. Hutcheon, I. D. Miller, I. C. Smith, and S. D. Heys, Monitoring primary breast cancer throughout chemotherapy using FDG-PET, Breast Cancer Res. Treat. 102 (2007) 75-84.
[37] R. Kumar, A. Chauhan, H. Zhuang, P. Chandra, M. Schnall, and A. Alavi, Standardized uptake values of normal breast tissue with 2-deoxy-2-[F-18]fluoro-D: -glucose positron emission tomography: variations with age, breast density, and menopausal status, Mol. Imaging Biol. 8 (2006) 355-362.
[38] N. Avril, C. A. Rose, M. Schelling, J. Dose, W. Kuhn, S. Bense, W. Weber, S. Ziegler, H. Graeff, and M. Schwaiger, Breast imaging with positron emission tomography and fluorine-18 fluorodeoxyglucose: use and limitations, J. Clin. Oncol. 18 (2000) 3495-3502.
[39] H. S. Lim, W. Yoon, T. W. Chung, J. K. Kim, J. G. Park, H. K. Kang, H. S. Bom, and J. H. Yoon, FDG PET/CT for the detection and evaluation of breast diseases: usefulness and limitations, Radiographics 27 Suppl 1 (2007) S197-S213.
[40] A. K. Karam and B. Y. Karlan, Ovarian cancer: the duplicity of CAl25 measurement, Nat. Rev. Clin. Oncol. 7 (2010) 335-339.
[41] A. Schrecengost, Ovarian mass—benign or malignant?, AORN J. 76 (2002) 792-796.
[42] G. H. Eltabbakh, P. R. Yadav, and A. Morgan, Clinical picture of women with early stage ovarian cancer, Gynecol. Oncol. 75 (1999) 476-479.
[43] E. Daoud and G. Bodor, CA-125 concentrations in malignant and nonmalignant disease, Clin. Chem. 37 (1991) 1968-1974.
[44] J. A. Sprowl, K. Reed, S. R. Armstrong, C. Lanner, B. Guo, I. Kalatskaya, L. Stein, S. L. Hembruff, A. Tam, and A. M. Parissenti, Alterations in tumor necrosis factor signaling pathways are associated with cytotoxicity and resistance to taxanes: a study in isogenic resistant tumor cells, Breast Cancer Res. 14 (2012) R2.
[45] J. A. DiDonato, F. Mercurio, and M. Karin, NF-kappaB and the link between inflammation and cancer, Immunol. Rev. 246 (2012) 379-400.
[46] G. Wu, X. Feng, and L. Stein, A human functional protein interaction network and its application to cancer data analysis, Genome Biol 11 (2010) R53.
[47] S. J. Clarke and L. P. Rivory, Clinical pharmacokinetics of docetaxel, Clin Pharmacokinet. 36 (1999) 99-114.
[48] C. Montagut, I. Tusquets, B. Ferrer, J. M. Corominas, B. Bellosillo, C. Campas, M. Suarez, X. Fabregat, E. Campo, P. Gascon, S. Serrano, P. L. Fernandez, A. Rovira, and J. Albanell, Activation of nuclear factor-kappa B is linked to resistance to neoadjuvant chemotherapy in breast cancer patients, Endocr. Relat Cancer 13 (2006) 607-616.
[49] A. M. Parissenti, J. A. Chapman, H. J. Kahn, B. Guo, L. Han, P. O'Brien, M. P. Clemons, R. Jong, R. Dent, B. Fitzgerald, K. I. Pritchard, L. E. Shepherd, and M. E. Trudeau, Association of low tumor RNA integrity with response to chemotherapy in breast cancer patients, Breast Cancer Res. Treat. 119 (2010) 347-356.
[50] P. Therasse, S. G. Arbuck, E. A. Eisenhauer, J. Wanders, R. S. Kaplan, L. Rubinstein, J. Verweij, G. M. Van, A. T. van Oosterom, M. C. Christian, and S. G. Gwyther, New guidelines to evaluate the response to treatment in solid tumors. European Organization for Research and Treatment of Cancer, National Cancer Institute of the United States, National Cancer Institute of Canada, J. Natl. Cancer Inst. 92 (2000) 205-216.
[51] R. Rouzier, C. M. Perou, W. F. Symmans, N. Ibrahim, M. Cristofanilli, K. Anderson, K. R. Hess, J. Stec, M. Ayers, P. Wagner, P. Morandi, C. Fan, I. Rabiul, J. S. Ross, G. N. Hortobagyi, and L. Pusztai, Breast cancer molecular subtypes respond differently to preoperative chemotherapy, Clin. Cancer Res. 11 (2005) 5678-5685.
[52] Koti, M., Vidal, R., Nuin, P., Haslehurst, A., Weberpals, J., Childs, T., Bryson, P., Feilloter, H. E., Squire, J., and Park, P. C. Identification of biomarkers of chemoresistance in serous epithelial ovarian cancer using integrative molecular profiling. Abstract 11-A-6026-AACR. American Association for Cancer Research 101st Annual Meeting. 2011. Ref Type Generic
[53] G. T. Sharma, P. K. Dubey, and G. S. Kumar, Effects of IGF-1, TGF-alpha plus TGF-beta(1) and bFGF on in vitro survival, growth and apoptosis in FSH-stimulated buffalo (Bubalis bubalus) preantral follicles, Growth Horm. IGF. Res. (2010).
[54] B. Wojciak-Stothard, A. Entwistle, R. Garg, and A. J. Ridley, Regulation of TNF-alpha-induced reorganization of the actin cytoskeleton and cell-cell junctions by Rho, Rac, and Cdc42 in human endothelial cells, J Cell Physiol 176 (1998) 150-165.
[55] A. J. Ridley, Rho proteins: linking signaling with membrane trafficking, Traffic. 2 (2001) 303-310.
[56] S. E. James, H. Burden, R. Burgess, Y. Xie, T. Yang, S. M. Massa, F. M. Longo, and Q. Lu, Anti-cancer drug induced neurotoxicity and identification of Rho pathway signaling modulators as potential neuroprotectants, Neurotoxicology 29 (2008) 605-612.
[57] T. Goto, M. Takano, M. Sakamoto, A. Kondo, J. Hirata, T. Kita, H. Tsuda, Y. Tenjin, and Y. Kikuchi, Gene expression profiles with cDNA microarray reveal RhoGDI as a predictive marker for paclitaxel resistance in ovarian cancers, Oncol. Rep. 15 (2006) 1265-1271.
[58] S. Sethu, G. Mendez-Corao, and A. J. Melendez, Phospholipase D1 plays a key role in TNF-alpha signaling, J. Immunol. 180 (2008) 6027-6034.
[59] L. Zernichow, M. Abrink, J. Hallgren, M. Grujic, G. Pejler, and S. O. Kolset, Serglycin is the major secreted proteoglycan in macrophages and has a role in the regulation of macrophage tumor necrosis factor-alpha secretion in response to lipopolysaccharide, J. Biol. Chem. 281 (2006) 26792-26801.
[60] Y. Z. Lin, S. Y. Yao, R. A. Veach, T. R. Torgerson, and J. Hawiger, Inhibition of nuclear translocation of transcription factor NF-kappa B by a synthetic peptide containing a cell membrane-permeable motif and nuclear localization sequence, J. Biol. Chem. 270 (1995) 14255-14258.
[61] C. Wahl, S. Liptay, G. Adler, and R. M. Schmid, Sulfasalazine: a potent and specific inhibitor of nuclear factor kappa B, J. Clin. lnvest 101 (1998) 1163-1174.
[62] J. Riondel, M. Jacrot, F. Picot, H. Beriel, C. Mouriquand, and P. Potier, Therapeutic response to taxol of six human tumors xenografted into nude mice, Cancer Chemother. Pharmacol. 17 (1986) 137-142.
[63] M. Mitsumoto, T. Kamura, H. Kobayashi, T. Sonoda, T. Kaku, and H. Nakano, Emergence of higher levels of invasive and metastatic properties in the drug resistant cancer cell lines after the repeated administration of cisplatin in tumor-bearing mice, J. Cancer Res. Clin. Oncol. 124 (1998) 607-614.
[64] B. A. Teicher, T. S. Herman, S. A. Holden, Y. Y. Wang, M. R. Pfeffer, J. W. Crawford, and E. Frei, III, Tumor resistance to alkylating agents conferred by mechanisms operative only in vivo, Science 247 (1990) 1457-1461.
[65] R. M. Tucker, R. J. Hendrickson, N. Mukaida, R. G. Gill, and C. L. Mack, Progressive biliary destruction is independent of a functional tumor necrosis factor-alpha pathway in a rhesus rotavirus-induced murine model of biliary atresia, Viral Immunol. 20 (2007) 34-43.
[66] B. L. Probst, L. Liu, V. Ramesh, L. Li, H. Sun, J. D. Minna, and L. Wang, Smac mimetics increase cancer cell response to chemotherapeutics in a TNF-alpha-dependent manner, Cell Death. Differ. 17 (2010) 1645-1654.
[67] S. Muerkoster, A. Arlt, M. Witt, A. Gehrz, S. Haye, C. March, F. Grohmann, K. Wegehenkel, H. Kalthoff, U. R. Folsch, and H. Schafer, Usage of the NF-kappaB inhibitor sulfasalazine as sensitizing agent in combined chemotherapy of pancreatic cancer, Int. J. Cancer 104 (2003) 469-476.
[68] B. Stoelcker, B. Ruhland, T. Hehlgans, H. Bluethmann, T. Luther, and D. N. Mannel, Tumor necrosis factor induces tumor necrosis via tumor necrosis factor receptor type 1-expressing endothelial cells of the tumor vasculature, Am. J. Pathol. 156 (2000) 1171-1176.
[69] S. Gupta, A decision between life and death during TNF-alpha-induced signaling, J Clin Immunol. 22 (2002) 185-194.
[70] M. Karin and E. Gallagher, TNFR signaling: ubiquitin-conjugated TRAFfic signals control stop-and-go for MAPK signaling complexes, Immunol. Rev 228 (2009) 225-240.
[71] M. A. gli-Esposti, W. C. Dougall, P. J. Smolak, J. Y. Waugh, C. A. Smith, and R. G. Goodwin, The novel receptor TRAIL-R4 induces NF-kappaB and protects against TRAIL-mediated apoptosis, yet retains an incomplete death domain, Immunity 7 (1997) 813-820.
[72] T. Hashimoto, D. Schlessinger, and C. Y. Cui, Troy binding to lymphotoxin-alpha activates NF kappa B mediated transcription, Cell Cycle 7 (2008) 106-111.
[73] S. A. Marsters, T. M. Ayres, M. Skubatch, C. L. Gray, M. Rothe, and A. Ashkenazi, Herpesvirus entry mediator, a member of the tumor necrosis factor receptor (TNFR) family, interacts with members of the TNFR-associated factor family and activates the transcription factors NF-kappaB and AP-1, J Biol Chem 272 (1997) 14029-14032.
[74] Y. Wu, D. Bressette, J. A. Carrell, T. Kaufman, P. Feng, K. Taylor, Y. Gan, Y. H. Cho, A. D. Garcia, E. Gollatz, D. Dimke, D. LaFleur, T. S. Migone, B. Nardelli, P. Wei, S. M. Ruben, S. J. Ullrich, H. S. Olsen, P. Kanakaraj, P. A. Moore, and K. P. Baker, Tumor necrosis factor (TNF) receptor superfamily member TACI is a high affinity receptor for TNF family members APRIL and BLyS, J Biol Chem 275 (2000) 35478-35485.
[75] P. Liston, N. Roy, K. Tamai, C. Lefebvre, S. Baird, G. Cherton-Horvat, R. Farahani, M. McLean, J. E. Ikeda, A. MacKenzie, and R. G. Korneluk, Suppression of apoptosis in mammalian cells by NAIP and a related family of IAP genes, Nature 379 (1996) 349-353.
[76] S. Scharf, S. Hippenstiel, A. Flieger, N. Suttorp, and P. D. N′ guessan, Induction of human {beta}-Defensin-2 in pulmonary epithelial cells by Legionella pneumophila: Involvement of TLR2 and TLR5, p38 MAPK, JNK, NF-{kappa}B and AP-1, Am J Physiol Lung Cell Mol Physiol (2010).
[77] T. R. Brummelkamp, S. M. Nijman, A. M. Dirac, and R. Bernards, Loss of the cylindromatosis tumour suppressor inhibits apoptosis by activating NF-kappaB, Nature 424 (2003) 797-801.
[78] G. Chen, M. Rong, and D. Luo, TNFRSF6B neutralization antibody inhibits proliferation and induces apoptosis in hepatocellular carcinoma cell, Pathol. Res. Pract. 206 (2010) 631-641.
[79] X. Li, M. Commane, H. Nie, X. Hua, M. Chatterjee-Kishore, D. Wald, M. Haag, and G. R. Stark, Act1, an NF-kappa B-activating protein, Proc Natl Acad Sci U. S. A 97 (2000) 10489-10493.
[80] T. D. Rachner, P. Benad, M. Rauner, C. Goettsch, S. K. Singh, M. Schoppet, and L. C. Hofbauer, Osteoprotegerin production by breast cancer cells is suppressed by dexamethasone and confers resistance against TRAIL-induced apoptosis, J. Cell Biochem. 108 (2009) 106-116.
[81] J. Bertin, L. Wang, Y. Guo, M. D. Jacobson, J. L. Poyet, S. M. Srinivasula, S. Merriam, P. S. DiStefano, and E. S. Alnemri, CARD11 and CARD14 are novel caspase recruitment domain (CARD)/membrane-associated guanylate kinase (MAGUK) family members that interact with BCL10 and activate NF-kappa B, J Biol Chem 276 (2001) 11877-11882.
[82] E. Lye, C. Mirtsos, N. Suzuki, S. Suzuki, and W. C. Yeh, The role of interleukin 1 receptor-associated kinase-4 (IRAK-4) kinase activity in IRAK-4-mediated signaling, J Biol Chem 279 (2004) 40653-40658.
[83] Y. Xu, X. Tao, B. Shen, T. Horng, R. Medzhitov, J. L. Manley, and L. Tong, Structural basis for signal transduction by the Toll/interleukin-1 receptor domains, Nature 408 (2000) 111-115.
[84] W. Liao, Q. Xiao, V. Tchikov, K. Fujita, W. Yang, S. Wincovitch, S. Garfield, D. Conze, W. S. EI-Deiry, S. Schutze, and S. M. Srinivasula, CARP-2 is an endosome-associated ubiquitin ligase for RIP and regulates TNF-induced NF-kappaB activation, Curr Biol 18 (2008) 641-649.
[85] C. J. Clarke, T. G. Truong, and Y. A. Hannun, Role for neutral sphingomyelinase-2 in tumor necrosis factor alpha-stimulated expression of vascular cell adhesion molecule-1 (VCAM) and intercellular adhesion molecule-1 (ICAM) in lung epithelial cells: p38 MAPK is an upstream regulator of nSMase2, J Biol Chem 282 (2007) 1384-1396.
[86] J. S. Rockel, J. C. Kudirka, A. J. Guzi, and S. M. Bernier, Regulation of Sox9 activity by crosstalk with nuclear factor-kappaB and retinoic acid receptors, Arthritis Res Ther. 10 (2008) R3.
[87] T. L. Liu, H. Shimada, T. Ochiai, T. Shiratori, S. E. Lin, M. Kitagawa, K. Harigaya, M. Maki, M. Oka, T. Abe, M. Takiguchi, and T. Hiwasa, Enhancement of chemosensitivity toward peplomycin by calpastatin-stabilized NF-kappaB p65 in esophageal carcinoma cells: possible involvement of Fas/Fas-L synergism, Apoptosis. 11 (2006) 1025-1037.
[88] T. G. Willis, D. M. Jadayel, M. Q. Du, H. Peng, A. R. Perry, M. bdul-Rauf, H. Price, L. Karran, O. Majekodunmi, I. Wlodarska, L. Pan, T. Crook, R. Hamoudi, P. G. Isaacson, and M. J. Dyer, Bcl10 is involved in t(1; 14)(p22;q32) of MALT B cell lymphoma and mutated in multiple tumor types, Cell 96 (1999) 35-45.
[89] C. Peng, Y. Y. Cho, F. Zhu, Y. M. Xu, W. Wen, W. Y. Ma, A. M. Bode, and Z. Dong, RSK2 mediates NF-{kappa}B activity through the phosphorylation of IkappaBalpha in the TNF-R1 pathway, FASEB J. 24 (2010) 3490-3499.
[90] C. B. Weldon, A. B. Scandurro, K. W. Rolfe, J. L. Clayton, S. Elliott, N. N. Butler, L. I. Melnik, J. Alam, J. A. McLachlan, B. M. Jaffe, B. S. Beckman, and M. E. Burow, Identification of mitogen-activated protein kinase kinase as a chemoresistant pathway in MCF-7 cells by using gene expression microarray, Surgery 132 (2002) 293-301.
[91] A. Kotlyarov, A. Neininger, C. Schubert, R. Eckert, C. Birchmeier, H. D. Volk, and M. Gaestel, MAPKAP kinase 2 is essential for LPS-induced TNF-alpha biosynthesis, Nat. Cell Biol. 1 (1999) 94-97.
[92] H. Walczak, M. A. gli-Esposti, R. S. Johnson, P. J. Smolak, J. Y. Waugh, N. Boiani, M. S. Timour, M. J. Gerhart, K. A. Schooley, C. A. Smith, R. G. Goodwin, and C. T. Rauch, TRAIL-R2: a novel apoptosis-mediating receptor for TRAIL, EMBO J. 16 (1997) 5386-5397.
[93] L. Wang, F. Du, and X. Wang, TNF-alpha induces two distinct caspase-8 activation pathways, Cell 133 (2008) 693-703.
[94] M. Ahmad, S. M. Srinivasula, L. Wang, R. V. Talanian, G. Litwack, T. Fernandes-Alnemri, and E. S. Alnemri, CRADD, a novel human apoptotic adaptor molecule for caspase-2, and FasL/tumor necrosis factor receptor-interacting protein RIP, Cancer Res. 57 (1997) 615-619.
[95] E. N. Tsitsikov, D. Laouini, I. F. Dunn, T. Y. Sannikova, L. Davidson, F. W. AIt, and R. S. Geha, TRAF1 is a negative regulator of TNF signaling. enhanced TNF signaling in TRAF1-deficient mice, Immunity. 15 (2001) 647-657.
[96] R. M. Rowlett, C. A. Chrestensen, M. Nyce, M. G. Harp, J. W. Pelo, F. Cominelli, P. B. Ernst, T. T. Pizarro, T. W. Sturgill, and M. T. Worthington, MNK kinases regulate multiple TLR pathways and innate proinflammatory cytokines in macrophages, Am. J. Physiol Gastrointest. Liver Physiol 294 (2008) G452-G459.
[97] Rothe M, Wong S C, Henzel W J, Goeddel D V, A novel family of putative signal transducers associated with the cytoplasmic domain of the 75 kDa tumor necrosis factor receptor. Cell 1994, 78:681-692.
[98] Hembruff S L, Villeneuve D J, Parissenti A M: The optimization of quantitative reverse transcription PCR for verification of cDNA microarray data. Anal Biochem 2005, 345:237-249.
[99] Brazma A, Hingamp P, Quackenbush J, Sherlock G, Spellman P, Stoeckert C, Aach J, Ansorge W, Ball C A, Causton H C, Gaasterland T, Glenisson P, Holstege F C, Kim I F, Markowitz V, Matese J C, Parkinson H, Robinson A, Sarkans U, Schulze-Kremer S, Stewart J, Taylor R, Vilo J, Vingron M: Minimum information about a microarray experiment (MIAME)-toward standards for microarray data. Nat Genet. 2001, 29:365-371.
[100] Eisenhart C: The assumptions underlying the analysis of variance. Biometrics 1947, 3:1-21.
[101] Tamhane A C, Dunlop DD: Statistics and Data Analysis: From Elementary to Intermediate. 1^{st} edition. Englewood Cliffs, N.J.: Prentice Hall; 2000: 473-474.
[102] Gross J L, Yellen J: Graph Theory and Its Applications. 1st edition, CRC Press; 1998.
[103] Enright A J, Van D S, Ouzounis C A: An efficient algorithm for large-scale detection of protein families. Nucleic Acids Res 2002, 30:1575-1584.
[104] Shannon P, Markiel A, Ozier O, Baliga N S, Wang J T, Ramage D, Amin N, Schwikowski B, Ideker T: Cytoscape: a software environment for integrated models of biomolecular interaction networks. Genome Res 2003, 13:2498-2504.

1. A method of evaluating cancer cell drug response (e.g. toxicity/resistance) to a taxane drug and/or anthracycline drug, the method comprising:
a. measuring a level of a TNF biomarker in a biological sample comprising the cancer cell after contacting the cancer cell with the taxane drug and/or anthracycline drug;
b. detecting a difference or a lack of difference in the level of the TNF biomarker compared to a control; and
c. or identifying the cancer cell as sensitive or resistant to the to taxane drug and/or anthracycline drug according to the detected difference in the TNF biomarker level.

2. The method of claim 1, wherein the TNF biomarker is selected from TNFα and active NFκB and the biomarkers listed in Table 2, wherein an increased level of TNFα compared to the control is indicative of toxicity to the taxane drug and/or anthracycline drug and an increased level of active NFκB compared to the control is indicative of resistance to the taxane drug and/or anthracycline drug.

3. The method of claim 1, wherein cancer cell is a breast cancer cell, an ovarian cancer cell, a sarcoma cell, a lymphoma cell, a leukemic cell, a uterine cancer cell, a colon cancer cell or a lung cancer cell.

4. The method of claim 1, wherein an increased level of a TNF resistance biomarker and/or decreased level in a TNF sensitivity biomarker compared to the control is indicative of resistance of the cancer cell to the taxane drug and an increased level of a TNF sensitivity biomarker and/or a decreased level of a TNF resistance biomarker compared to the control is indicative of toxicity/sensitivity of the cancer cell to the taxane drug and/or anthracycline drug, optionally wherein the TNF resistance biomarker is active NFkappaB, TNFR2 and/or a biomarker listed as increased in Table 2 and/or the TNF sensitivity biomarker is TNFalpha, TNFR1 or a biomarker listed as decreased in Table 2.

5. The method of claim 1, wherein the control is a selected reference threshold value derived from a population of nonresistant cells and/or a population of resistant cells, wherein an increased level of a resistance TNF biomarker and/or a decreased level of a sensitive TNF biomarker are associated with taxane and/or anthracycline resistance or a decreased level of a resistance TNF biomarker and/or an increased level of a sensitive TNF biomarker are associated with taxane and/or anthracycline sensitivity.

6. The method according to claim 1 wherein the method comprises: contacting the cancer cell with the taxane drug and/or anthracycline drug; prior to measuring the level of the TNF biomarker in the biological sample comprising the cancer cell.

7. The method of claim 1, wherein the cancer cell is a primary cancer cell, optionally in vitro or in vivo.

8. A method of evaluating cancer cell response to administration of a taxane drug and/or anthracycline drug in a subject in need thereof, the method comprising:
a. measuring a level of a TNF biomarker in a biological sample comprising pathologic tissue obtained from the subject after administering to the subject of one or more doses of the taxane drug and/or anthracycline drug;
b. detecting a difference or a lack of difference in the relative level of the TNF biomarker compared to a control; and
c. identifying the cancer cell as sensitive or resistant to the resistance to taxane drug and/or anthracycline drug according to the detected difference in in the TNF biomarker level.

9. The method of claim 8, wherein the method comprises administering one or more doses of the taxane drug and/or anthracycline drug to the subject prior to measuring the level of the TNF biomarker.

10. The method of claim 9, wherein the TNF biomarker is TNFα.

11. The method of claim 8 for evaluating clinical response and/or predicting clinical response in a subject afflicted with breast or ovarian cancer, the method comprising:
a. measuring a level of a TNF biomarker, such as TNFα, in a biological sample comprising pathologic tissue obtained from the subject after administering to the subject one or more doses of the taxane drug and/or anthracycline drug;
b. detecting a difference or lack of difference in the relative level of the TNF biomarker compared to a control; and
c. predicting the clinical response for the subject according to the detected difference in in the relative leve of the TNF biomarker;
wherein i) an increase in a relative level of TNFα and/or a TNF sensitivity biomarker is indicative that the subject is positively responding and/or will have a positive clinical response or ii) a lack of increase or a decrease in a relative level of TNFα and/or a TNF sensitivity biomarker and/or an increase in a relative level of a TNF resistance biomarker is indicative that the subject is negatively responding and/or will have a poor clinical response.

12. The method of claim 11 wherein the method comprises administering one or more doses of a taxane drug and/or anthracycline drug to the subject prior to measuring the level of the TNF biomarker.

13. The method of claim 12, wherein the drug is administered systemically or directly to the tumour.

14. The method of claim 11, wherein the TNF resistance biomarker is selected from active NFkappaB, TNFR2 and/or a biomarker listed as increased in Table 2, optionally BIRC3, TLR6 and/or TNFSF10 and/or the TNF sensitivity biomarker is selected from TNFalpha, TNFR1 or a biomarker listed as decreased in Table 2.

15. The method of claim 11, wherein the clinical response is progressive disease, stable disease, partial response, complete clinical response or pathological complete response.

16. The method of claim 11, wherein the relative level of the TNF biomarker, optionally TNFalpha, is increased at least 2×, 3×, 4×, 5×, 6×, 7×, 8×, 9×, 10×, 11×, 12×, 13×, 14×, 15×, 16×, 17×, 18×, 19×, or at least 20× compared to control.

17. The method of claim 11, wherein the breast cancer is locally advanced breast cancer (LABC) or inflammatory breast cancer, and/or the breast cancer is Her2+, triple negative, basal subtype, luminal A, normal, or luminal B subtype (e.g. luminal B1 or luminal b2), unclassified Her1+ve. optionally invasive breast canceroptionally wherien the histologic type of invasive breast cancer is invasive ductal carcinoma, invasive lobular carcinoma, medullary carcinoma or tubular carcinoma, optionally wherein the grade is grade I, II or III and/orhe ovarian cancer is epithelial, serous, mucinous, endometrioid, clear cell, or undifferentiated/unclassified ovarian cancer.

18. The method of claim 11, wherein the taxane drug is selected from paclitaxel, docetaxel, larotaxel, Abraxane, docoxahexaenoic acid-linked paclitaxel, paclitaxel polyglumex, Ortataxel, Genexol, liposomal-encapsulated paclitaxel, and paclitaxel in a Vitamin E emulsion and/or wherein, the anthracycline drug is selected from epirubicin, doxorubicin, epirubicin, daunorubicin, idarubicin, valrubicin, and mitoxantrone.

19. The method of claim 11, wherein the TNF biomarker measured is TNF biomarker transcript or TNF biomarker polypeptide. optionally wherein the TNFalpha transcript is measured by polymerase chain reaction (PCR).

20. The method of claim 19, wherein a relative TNF alpha transcript level is determined, relative to one or more reference standard gene expression levels.

21. The method of claim 20, wherein, the one or more reference standard genes are selected from HMBS, HPRT1, MRPL19, PUM1, RPL13A, SDHA, and SF3A1, optionally 3 or more, 4 or more, 5 or more 6 or more or all 7 of said reference genes, optionally wherein the level of said reference genes is measured using a primer set for the corresponding reference standard gene listed in Table 3.

22. The method of claim 11, wherein, the level of the TNF biomarker is assessed mid-treatment or post-treatment.

23. A method of treating a subject afflicted with breast or ovarian cancer, the method comprising:
a. administering one or more doses of a taxane drug and/or anthracycline drug treatment to the subject;
b. and/or identifying the cancer cell as sensitive or resistant to the and/or predicting clinical response in a subject afflicted with breast or ovarian cancer according to a method described herein, optionally claim 11; and
c. continuing the taxane drug and/or anthracycline drug treatment when the cancer cell is determined to be responsive and/or when the clinical response determined is a good clinical outcome or discontinuing the taxane drug and/or anthracycline drug treatment when the cancer cell is determined to be resistant and/or when the clinical response is determined to be a poor clinical outcome.

24. The method of claim 23, wherein the subject is also treated with an agent selected from a TNFR1 agonist and a TNFR2 antagonist.

25. The method of claim 11, comprising:
a. optionally administering one or more doses of a taxane drug and/or anthracycline drug to the subject;
b. measuring a level of a TNFα in a biological sample comprising pathological tissue obtained from the subject after administration of the one or more doses of the taxane drug and/or anthracycline drug; and
c. detecting a difference in the relative level of the TNFα compared to a control;
wherein i) an increase in the relative level of TNFα is indicative that the subject is positively responding and/or will have a positive clinical response or ii) a lack of increase or decrease in the relative level of the TNFα is indicative that the subject is negatively responding and/or will have a poor clinical response.

26. A method for predicting response in a patient by analyzing a subject sample for the presence or absence of a taxane and/or anthracycline drug sensitive cancer cell by measuring the level of one or more TNF biomarkers, wherein the subject is predicting to be responding to the taxane and/or anthracycline drug if an increased level of a TNF sensitive biomarker and/or a decreased level of a TNF resistance biomarker is detected.


SEQUENCE LISTING (36 sequences)

SEQ ID NO: 1 - DNA, 21 bp, Homo sapiens
actgcctcagctgctccaaat

SEQ ID NO: 2 - DNA, 19 bp, Homo Sapiens
ccggtccactgtgcaagaa

SEQ ID NO: 3 - DNA, 20 bp, Homo Sapiens
tcttctcgaaccccgagtga

SEQ ID NO: 4 - DNA, 18 bp, Homo Sapiens
ggagctgcccctcagctt

SEQ ID NO: 5 - DNA, 21 bp, Homo Sapiens
tccatcatccgcaatgtaaag

SEQ ID NO: 6 - DNA, 21 bp, Homo Sapiens
gcttctcgctctgactccaaa

SEQ ID NO: 7 - DNA, 22 bp, Homo Sapiens
gaccatggcacaactcatctca

SEQ ID NO: 8 - DNA, 28 bp, Homo Sapiens
gttagcttcatccaactttgcggcattg

SEQ ID NO: 9 - DNA, 23 bp, Homo Sapiens
cgtgtactttaccaacgagctga

SEQ ID NO: 10 - DNA, 21 bp, Homo Sapiens
acggagttgccacttgacttg

SEQ ID NO: 11 - DNA, 22 bp, Homo Sapiens
actctcagttgccctctggttg

SEQ ID NO: 12 - DNA, 22 bp, Homo Sapiens
ggaactctgctccgggagactc

SEQ ID NO: 13 - DNA, 23 bp, Homo Sapiens
tttgctccacagttggcctaatc

SEQ ID NO: 14 - DNA, 22 bp, Homo Sapiens
caatgactgtggcctcaccttc

SEQ ID NO: 15 - DNA, 25 bp, Homo Sapiens
ggtaccaggccctcttcctcgttag

SEQ ID NO: 16 - DNA, 25 bp, Homo Sapiens
taggaacgtggatgagaccgttttt

SEQ ID NO: 17 - DNA, 25 bp, Homo Sapiens
gcaaaaacccttcaccttgtttttc

SEQ ID NO: 18 - DNA, 25 bp, Homo Sapiens
ccaagtcgtttctatgtggttgagg

SEQ ID NO: 19 - DNA, 20 bp, Homo Sapiens
tgttgggaatctggagatga

SEQ ID NO: 20 - DNA, 20 bp, Homo Sapiens
cggatgaactcctgtccttt

SEQ ID NO: 21 - DNA, 21 bp, Homo Sapiens
gcttcaccatcggagccatct

SEQ ID NO: 22 - DNA, 21 bp, Homo Sapiens
tggcagggtttctagggtctt

SEQ ID NO: 23 - DNA, 21 bp, Homo Sapiens
tgacactggcaaaacaatgca

SEQ ID NO: 24 - DNA, 21 bp, Homo Sapiens
ggtccttttcaccagcaagct

SEQ ID NO: 25 - DNA, 28 bp, Homo Sapiens
aaggagaaaagtactccacattccagag

SEQ ID NO: 26 - DNA, 22 bp, Homo Sapiens
tgggtcagctgtagtaacacga

SEQ ID NO: 27 - DNA, 19 bp, Homo Sapiens
tgaggtgtgcaccatgaac

SEQ ID NO: 28 - DNA, 20 bp, Homo Sapiens
cagaatgtgcttgccatagg

SEQ ID NO: 29 - DNA, 23 bp, Homo Sapiens
cctggaggagaagaggaaagaga

SEQ ID NO: 30 - DNA, 25 bp, Homo Sapiens
ttgaggacctctgtgtatttgtcaa

SEQ ID NO: 31 - DNA, 22 bp, Homo Sapiens
tgttgatgggaacaagagggca

SEQ ID NO: 32 - DNA, 22 bp, Homo Sapiens
gcctaccaccactgcatcaaat

SEQ ID NO: 33 - DNA, 21 bp, Homo Sapiens
ggaggattctgcaccttctaa

SEQ ID NO: 34 - DNA, 19 bp, Homo Sapiens
gcggtagtaggcatggtaa

SEQ ID NO: 35 - DNA, 20 bp, Homo Sapiens
tgcctgctgcactttggagt

SEQ ID NO: 36 - DNA, 21 bp, Homo Sapiens
tgggccagagggctgattaga